(19) United States
(12) Patent Application Publication
(10) Pub. No.: US 2020/0075167 A1
(43) Pub. Date: Mar. 5, 2020

(54) DYNAMIC ACTIVITY RECOMMENDATION SYSTEM

(71) Applicant: Regents of the University of Minnesota, Minneapolis, MN (US)

(72) Inventors: Jaideep Srivastava, Minneapolis, MN (US); Aarti Sathyanarayana, Minneapolis, MN (US)

(21) Appl. No.: 16/556,647

(22) Filed: Aug. 30, 2019

Related U.S. Application Data
(60) Provisional application No. 62/724,975, filed on Aug. 30, 2018.

(51) Int. Cl. G16H 50/20

(52) U.S. Cl. CPC G16H 50/20

(57) ABSTRACT

A method includes associating a person's activities up to a current time point with one of a plurality of clusters of activity proportions for the current time point and associating the person's activities up to the current time point with a sub-cluster in the cluster of activity proportions for the current time point. A determination is then made that the sub-cluster is associated with poor sleep and a recommendation of at least one activity to increase the likelihood of good sleep is made.

### CROSS-REFERENCE OF RELATED APPLICATION

The present application is based on and claims the benefit of U.S. provisional patent application Ser. No. 62/724,975, filed Aug. 30, 2019, the content of which is hereby incorporated by reference in its entirety.

### BACKGROUND

By the mid-1900s, sleep laboratories began to appear that were dedicated to studying sleep-related disorders such as restless legs syndrome, sleep-obstructive apnea, narcolepsy, and insomnia. These labs resulted in more systematic sleep studies and the development of polysomnography (PSG), a sleep-study methodology that incorporates various sensors to capture brain activity, leg movement, oxygen saturation, breathing frequency, and snoring. [M. Hirshkowitz, “The history of polysomnography: Tool of scientific discovery,” Sleep Medicine: A Comprehensive Guide to Its Development, Clinical Milestones, and Advances in Treatment, pp. 91-100, 2015] Although it is a major diagnostic tool that is considered the gold standard for sleep research, PSG is performed only in sleep laboratories in hospitals. During a PSG test, the patient spends at least one night in the unnatural environment of a sleep laboratory monitored by a clinician and attached to multiple sensors. On one hand, PSG is a high-fidelity test; on the other hand, it is not scalable, too expensive, does not consider physical activity (which is tightly linked to sleep quality), and does not provide a holistic window into a patient behaviours in their natural everyday environment. As a result, PSG cannot keep pace with the growing number of sleep disorders, and large segments of the population still suffer from inadequate sleep.

More recently, Home Sleep Tests (HSTs) have been proven as an effective alternative to PSG [N. J. Douglas, “Home diagnosis of the obstructive sleep apnoea/hypopnoea syndrome,” Sleep medicine reviews, vol. 7, no. 1, pp. 53-59, 2003]. HSTs still require an in clinic visit with a specialist, but rather than spending the night at a clinic, patients take home a small device. The device consists of a small nasal cannula to measure airflow, a chest belt to measure respiration, and a finger clip to measure the blood oxygen saturation. Since HSTs use far fewer sensors than PSG, they are limited to diagnosing only sleep apnea. Whilst they are a much more affordable alternative, the lack of clinical monitoring or computational automation, means that HSTs make no distinction between awake time and sleep time. This leads to sleep apnea being under-diagnosed, particularly in patients that are slow to fall asleep.

The discussion above is merely provided for general background information and is not intended to be used as an aid in determining the scope of the claimed subject matter. The claimed subject matter is not limited to implementations that solve any or all disadvantages noted in the background.

### SUMMARY

A method includes associating a person's activities up to a current time point with one of a plurality of clusters of activity proportions for the current time point and associating the person's activities up to the current time point with a sub-cluster in the cluster of activity proportions for the current time point. A determination is then made that the sub-cluster is associated with poor sleep and a recommendation of at least one activity to increase the likelihood of good sleep is made.

In accordance with a further embodiment, a method includes clustering partial activity histograms, wherein each partial activity histogram extends from a start of activities to a selected time after the start of activities. For each cluster, sub-clusters of full activity histograms are formed, wherein each full activity histogram extends from a start of activities to an end of activities. For each sub-cluster, a most-likely outcome of the full activity histograms in the sub-cluster is identified. An in-progress activity histogram extending from the start of activities to the selected time after the start of activities is received and a most-likely outcome for the in-progress activity histogram is identified by assigning the in-progress activity histogram to one of the sub-clusters.

In accordance with a still further embodiment, a system includes an activity sensor providing periodic activity values and a sleep prediction module, predicting at multiple times per day a quality of sleep that is expected at the end of the day based on the periodic activity values.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is a simplified combined flow diagram and block diagram in accordance with one embodiment.

FIG. 2 is a sample accelerometer output with sleep definitions annotated.

FIG. 3 is a Actigraph GTX3+.

FIG. 4 is an automated Actigraphy state machine.

FIG. 5 is a sample accelerometer output used to predict sleep quality in accordance with one embodiment.

FIG. 6 is a multi-layer perceptron with one hidden layer.

FIG. 7 is a convolutional neural network.

FIG. 8 is a recurrent neural network with one recurrent layer.

FIG. 9 is an LSTM memory block.

FIG. 10 is a time-batched long short-term memory cell recurrent neural network architecture.

FIG. 11 is ROC curves of logistic regression.

FIG. 12 is a sample activity feature set used to predict sleep quality.

FIG. 13 is an illustration of the RAHAR workflow.

FIG. 14 is a high-level overview of RAHAR.

FIG. 15 is a visualization of change point detection using Hierarchical divisive estimation.

FIG. 16 is an illustration of change points and change point intervals.

FIG. 17 is ROC curves for RAHAR

FIG. 18 is ROC curves for sleep expert using ActiLife software.

FIG. 19 is a comparison of the ROC for RAHAR and SE+AL on the best model.

FIG. 20 is a comparison of sensitivity and specificity for RAHAR and SE+AL on the best model.

FIG. 21 is a tree outlining all possible prediction and reality combinations.

FIG. 22 is a confusion matrix of predicted sleep quality, behavior upon recommendation and real sleep quality.

FIG. 23 is a visualization of sub-clustering.

FIG. 24 is a visualization of the Calinski-Harabasz Index used to determine the ideal number of clusters.

FIG. 25 is a visualization of the clustering in parallel coordinates with 10 clusters.

FIG. 26(a)-(t) is a visualization of the subcluster size selection using the Calinski criterion, and the cluster centroids using parallel coordinates.

FIG. 27 is a confusion matrix with results from a retrospective analysis.

FIG. 28 is a block diagram of a system for predicting sleep quality and making activity recommendations in accordance with one embodiment.

FIG. 29 is a block diagram of a computing environment used in accordance with the various embodiments.

### DETAILED DESCRIPTION

Wearable devices provide the first hope of solving the problems associated with PSG and HST because they can be used to study sleep and physical activity for longer periods of time and outside the laboratory or hospital. Because a wearable device monitors the patient's body continuously, collected signals can be more effectively used to determine sleep quality and screen for sleep disorders.

Actigraphy devices, such as the GT3X from ActiGraph, are clinical-grade wearables approved for sleep studies that use inertial sensors to collect physical activity and sleep data. As of January 2017, actigraphy was being used in more than 100 clinical trials registered in the US clinical trials database (www.clinicaltrials.gov). In many applications, actigraphy can be a cheaper and simpler alternative to PSG. In fact, actigraphy devices are already advancing sleep science in areas such as obesity, diabetes, cancer, mental health, and public health.

Sleep analytics is no longer restricted to researchers and clinicians, as the pervasiveness of mobile health and wellness applications attests. Millions of people use affordable wearables to track their physical activity and sleep, while companion applications integrate tracked data into mobile health repositories, such as Apple HealthKit. These repositories enable integration with other medical devices, such as heart rate monitors, and even with electronic health records (EHRs). The Apple Watch has an instrument suite to collect data on various bodily functions, including triaxial movement and heart rate. The watch seamlessly syncs with iOS devices, such as iPhones and iPads. With appropriate permissions, Apple HealthKit API allows queries to the collected data. Other smart-device manufacturers, such as Samsung and Microsoft, have created wearable platforms based on their respective operating systems. Specialized wearable device companies like Fitbit have developed their own platforms. Along with the myriad of fitness trackers and smart watches that can monitor sleep, are context sensors such as SleepScore's Max device (http://www.sleepscore.com/sleepscore-max-sleep-tracker/), Nokia's newly acquired Withings device Aura (www.withings.com/ca/en/products/aura/sleep-sensor-accessory), or Apple's acquisition of Beddit (www.beddit.com). Both include a sensing mat for sleep monitoring, and Aura integrates with the Nest Internet of Things (IoT) platform (www.nest.com) to automatically adjust room temperature for optimal sleep quality.

The importance of data for health research has become mainstream in recent years, and sleep science is no exception. The National Institutes of Health (NIH) created the National Sleep Research Resource [D. A. Dean, A. L. Goldberger, R. Mueller, M. Kim, M. Rueschman, D. Mobley, S. S. Sahoo, C. P. Jayapandian, L. Cui, M. G. Morrical et al., “Scaling up scientific discovery in sleep medicine: The national sleep research resource,” 2016.; R. Budhiraja, R. Thomas, M. Kim, and S. Redline, “The role of big data in the management of sleep-disordered breathing,” Sleep medicine clinics, vol. 11, no. 2, pp. 241-255, 2016], a portal aimed at integrating heterogeneous data sources for clinical sleep research (www.sleepdata.org). The portal, part of the well-known Big Data to Knowledge (BD2K) initiative, contains a wide variety of datasets for sleep research.

The current sleep analysis processes for actigraphy data include manual components that are unable to scale, creating a bottleneck for sleep research. Moreover, this leaves the data interpretation prone to human error. Particularly for population screening, it is critical that the methods and knowledge extracted are generalizable across populations (and devices) and thus robust to noise and variance amongst sub-populations. For example, teenagers in Qatar follow very different sleep patterns to teenagers in the UK. For computational models to effectively extract actionable medical knowledge, it is critical that the models are interpretable by health professionals. Without actionable insights, society cannot benefit from sleep research discoveries. Furthermore, automation improves consistency across individuals and datasets.

Data visualization is a further sub-speciality that is needed for the interpretability of the data. Presenting real-time insights to wearable device users can influence their behaviour and guide them towards a healthier lifestyle. Sharing information clearly with clinical professionals can allow doctors to quickly identify poor habits or identify more serious medical disorders, such as sleep apnea.

### Current Sleep Science Process

### Screening

Widespread sleep disorder screening has not yet been feasible, due to a lack of automation. Therefore current sleep science tools do not differentiate between screening and diagnosis. Sleep problems are self-reported by patients, appointments with a sleep specialist require a referral from a primary care physician, and any type of intervention or analysis, requires active involvement from a specialist. As a result, sleep clinics are overbooked and there is a long waitlist causing a bottleneck in patients receiving a formal diagnosis. Moreover, the costs incurred from a sleep clinic visit can be very expensive.

### Diagnosis

The gold standard for clinical sleep diagnosis is Polysomnography (PSG). As previously mentioned, PSG is a diagnostic tool which incorporates multiple channels from various sensors. It must be performed at a sleep laboratory in a hospital, or at a clinic, and a patient must spend at least one night monitored continuously by the clinician and attached to multiple sensors to track: electroencephalography, leg movement, oxygen saturation, breathing frequency, microphones for snoring, etc [R. J. Cole, D. F. Kripke, W. Gruen, D. J. Mullaney, and J. C. Gillin, “Automatic sleep/wake identification from wrist activity,” Sleep, vol. 15, no. 5, pp. 461-469, 1992].

PSG is a challenging and expensive solution. Although it provides a high-fidelity test, it cannot scale to tackle the growing prevalence of sleep disorders. Furthermore, PSG cannot consider other behaviours, such as physical activity, that are highly linked to sleep quality. This is a major limitation of PSG on providing insights into the behaviours of patients within their natural environment and daily routines. As a result of these shortcomings, sleep disorders often continue without diagnosis or therapy, leading to large segments of the population suffering from inadequate sleep. It is from this context that sleep science has been driven towards the use of wearable devices.

Actigraphy is another diagnostic technique. It is specifically useful for the extended study of sleep and physical activity patterns using clinical-grade wearable devices. The devices utilize inertial sensors such as accelerometers or inclinometers, to track behaviour and activity levels. An example is the GT3XÂ® from ActiGraph Corp, which is a medical actigraph device clinically validated for sleep studies.

Actigraphy, in many applications, can be a cheaper and simpler alternative to PSG, and is facilitating the advancement of sleep science in areas such as, obesity, diabetes, cancer, mental health and public health. Currently, actigraphy is used in over 100 clinical trials registered in the US Clinical Trials Database (clinicaltrials.gov). Data analytics has been widely used to study sleep patterns from actigraphy data [9-12], but primarily to study different sleep patterns and characteristics [M. Enomoto, T. Endo, K. Suenaga, N. Miura, Y. Nakano, S. Kohtoh, Y. Taguchi, S. Aritake, S. Higuchi, M. Matsuura et al., “Newly developed waist actigraphy and its sleep/wake scoring algorithm,” Sleep and Biological Rhythms, vol. 7, no. 1, pp. 17-22, 2009; N. Ravi, N. Dandekar, P. Mysore, and M. L. Littman, “Activity recognition from accelerometer data,” in Aaai, vol. 5, no. 2005, 2005, pp. 1541-1546; J. Baek, G. Lee, W. Park, and B.-J. Yun, “Accelerometer signal processing for user activity detection,” in Knowledge-Based Intelligent Information and Engineering Systems. plus 0.5em minus 0.4em Springer, 2004, pp. 610-617; A. Sathyanarayana, F. Ofli, L. Luque, J. Srivastava, A. Elmagarmid, T. Arora, and S. Taheri, “Robust automated human activity recognition and its application to sleep research,” in 2016 IEEE International Conference on Data Mining Workshop (ICDMW), December 2016].

In addition to tablets, there are generalized guidelines that are suggested to improve sleep quality via lifestyle changes. Relaxation techniques such as yoga, meditation, hynotherapy, massage therapy, acupuncture and aromatherapy are all thought to improve the quality of sleep. Improved sleep hygiene (i.e no use of blue light devices such as a phone/TV/computer before bed, sleeping in an entirely dark environment, only engaging in mild activity before bed, following a regular sleep schedule including on weekends etc.) also help the quality of sleep. There are also dietary guidelines such as staying away from caffeine, alcohol and heavy meals. These guidelines are made for the overall population and are not personalized to an individual. What is more, these guidelines are often not based on strong evidence obtained through transparent research or registered clinical trials.

Cognitive behavioural therapy (CBT) is a clinical technique used specifically for treating insomnia. It has been proven effective in coaching individuals towards an improved lifestyle for better sleep. A therapist or sleep expert works with a patient over a number of weeks to improve their habits. However, gaining access to CBT cohorts can often be inaccessible or a bottleneck to treatment. Not to mention, CBT requires extensive involvement from a professional.

### Proposed Revised Sleep Science Process

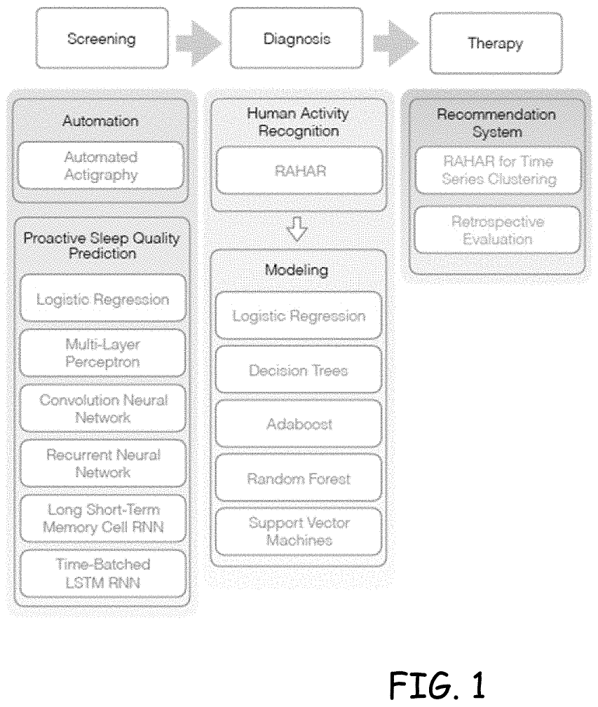

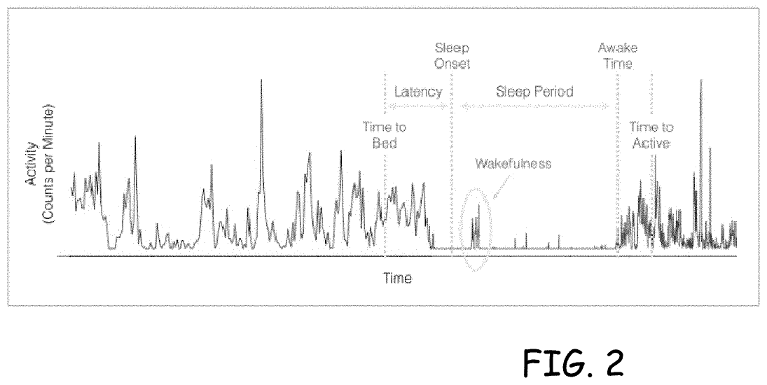

FIG. 1 shows computer science data-driven approaches to a sleep process pipeline. These methods take advantage of (i) the big data available in sleep science clinical trials and wearable devices, (ii) the cheap relative cost of data storage and computation, and (iii) the public acceptance of advanced “black box” algorithms.

Actigraphy currently requires the presence of a clinical professional to manually annotate and review the output. This is a huge bottleneck for evaluating clinical trial research, causes added expense and overhead time for clinic visits, and eliminates any possibility of widespread sleep problem screening. Thus automated actigraphy is a fundamental necessity to the sleep research community and allows for sleep quality evaluation on an individual's sleep period for wearable devices that are worn to bed. Section 2.2 dives into the details of this tool.

### Screening

While automated actigraphy is a critical tool to sleep researchers, wearables often need charging overnight (e.g. Apple Watch). Even more importantly, actigraphy is a retrospective evaluation of activity, i.e. it allows for sleep quality evaluation only after the fact. The various embodiments use data mining and machine learning to provide a prediction framework, so that sleep quality can be determined beforehand. This opens the door to the prevention of poor sleep, rather than the deduction. Classic statistical modeling methods such as linear (numerical) or logistic (classification) regression can be used for prediction. Data mining methods such as decision trees, random forest and support vector machines can also be used.

Deep learning has been a hugely popular methodology in the computer science industry over the last few years. The mathematical nature of deep learning makes it ideal for high-fidelity screening of sleep disorders. Models such as the universal approximater, a multi-layer perceptron can be used, as well as convolution neural networks, recurrent neural networks, and long short-term memory cells. In addition, embodiments provide new deep learning architectures that are designed specifically with the nature of wearable actigraphy data in mind.

### Diagnosis

Although deep learning has high predictive value, the justification of its prediction is not transparent. Deep learning has no beta coefficient equivalent. On the contrary, traditional statistical and data mining methods do provide insight into the prediction. The fact that traditional methods suffer in accuracy relative to deep learning, is due to the complex shape and nature of data. However, this complexity can be managed by conducting feature construction on the raw wearable device data. These modified learning representations of the raw data can improve downstream analysis and lead to improved results from traditional data analysis methods, and improved transparency relative to deep learning algorithms.

Human Activity Recognition (HAR) algorithms aim to classify and label human behaviour, from data captured by pervasive sensors, such as cameras or wearable devices. These algorithms are a powerful tool when consistent and continuous patient monitoring results in large longitudinal data collection. Embodiments utilize a human activity recognition on the raw accelerometer output to create a coarser feature space that significantly improves the capabilities of traditional data mining methods.

### Therapy

Lastly, embodiments provide tools that can be used within a real-time recommendation system. This recommendations system is used as a sleep coach within either (i) an application for consumer health fanatics to self-improve their sleep quality, or (ii) an automated alternative, or assistant, to cognitive behavioural therapy interventions. By evaluating an individuals daily activities, using human activity recognition, we can identify behavioural recipes that lead to good or poor quality sleep. These recipes can then be used as target behaviour for users throughout the day, to obtain a good night sleep. In order to identify the behavioural recipes, embodiments use clustering on a time series constructed from a human activity recognition alphabet.

### Sleep Science Metrics and Terminology

Throughout the remaining sections, domain specific terminology from the sleep science community is used. To clarify this vocabulary for the reader, these terms are defined in this section. FIG. 2 contains an annotated version of a sample activity time series collected from a wearable device.

Actigraphy: As aforementioned, actigraphy is the study of sleep-related behaviour measured via a clinically-validated wearable device. It is used as a tool to gain insights into an individual's physical activity and its effect of their sleep.

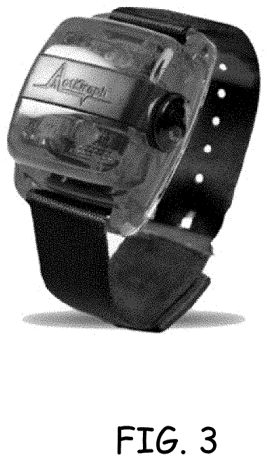

Actigraph: An actigraph is a clinically-validated wearable device. These devices contain an accelerometer, an inclinometer, and often other sensors such as a heart rate sensor, luminosity sensor, etc. The distinction between an ordinary consumer wearable and an actigraph, is in the validation. These devices are used as a clinical diagnostic tool, and for data collection in clinical trials. They are often tied to a software suite of tools for clinicians to conduct analysis. FIG. 3 is an image of an actigraph from the company Actigraph. The device is called an Actigraph GT3X+, and contains an accelerometer, inclinometer and lumosity sensor to measure ambient light.

Activity Time Series: The output from a wearable device includes the accelerometer movement, inclinometer movement, and any other sensor information the device contains, tracked over a continuous period of time. From the accelerometer output, counts are computed to represent the frequency and intensity of the raw acceleration in epochs. FIG. 2 graphs an activity time series, with time on the x-axis and counts per minute on the y-axis. Higher counts represent more intense movement.

Time to Bed: The time to bed is the time that an individual attempts to fall asleep. In traditional sleep science, this value is self-reported, and is thus often unreliable and inaccurate. Below, an automated actigraphy approach to determining the time to bed is described.

Sleep Onset Time: As opposed to time to bed, the sleep onset time is not self reported. It it the point in time where an individual actually falls asleep. In traditional actigraphy, the sleep onset time is calculated as the first minute following the self-reported time to bed, that precedes 15 continuous minutes of sleep, i.e. minimal movement tracked by the accelerometer. It also marks the start of the sleep period.

Latency: The difference in time between time to bed, and the sleep onset time, is referred to as the latency. In other words, latency is the amount of time it takes for an individual to fall asleep after they intend to. Whilst sleep quality is objectively measured through a variety of metrics, latency is often a good measure of the perceived sleep quality.

Awake Time: Once an individual is asleep, the moment in time that they awaken is referred to as the awake time. In traditional actigraphy, this value is self-reported.

Sleep Period: The sleep period is the specific period in time that is indexed by the sleep onset time and the time that an individual awakens.

Time to Active: Whilst the awake time is self-reported, time to active is computed from the actigraphy signal. More details on how this value is computed are included below.

Wakefulness and Wake After Sleep Onset: Accelerometers within actigraph devices, are very sensitive to movement. This is so that sedentary behaviour can be differentiated from actual sleep. However, when an individual is asleep, there is often also some small movement. Any periods of movement that are continuous for over 5 minutes, constitute wakefulness. The sum of all such periods of wakefulness during a sleep period, is called the wake after sleep onset, or WASO.

$$WASO = {\sum }_{{Sleep}_{{Onset}_{Time}}}^{{Sleep}_{{Awakening}_{Time}}} \{ \begin{matrix}‖ WakefulPeriod ‖ , & if ‖ WakefulPeriod ‖ , > 5 \\ 0 , & otherwise\end{matrix} \qquad ( 1.1 )$$

Sleep Efficiency: In sleep science, sleep quality is defined by a number of metrics, including total sleep time, wake after sleep onset, awakening index, and sleep efficiency [Normative values of polysomnographic parameters in childhood and adolescence: Quantitative sleep parameters,” Sleep Medicine, vol. 12, no. 6, pp. 542-549, 2011]. There are also questionnaires that strive for a more subjective understanding of sleep quality. Since sleep efficiency combines many of the aforementioned metrics, it is generally accepted as the best objective measure of sleep quality. Thus, embodiments treat sleep efficiency and sleep quality as synonymous. Sleep efficiency is computed as a numerical value ranging from 0 to 1. It is the ratio of total sleep time to total minutes in bed, i.e. the ratio of the length of the sleep period less the time spent awake, to the length of the sleep period plus the latency. According to specialists, a sleep efficiency below 0.85 (i.e., 85%) indicates poor sleep quality, and above 0.85 indicates good sleep quality [R. Williams, I. Karacan, and C. Hursch, Electroencephalography (Eeg) of Human Sleep: Clinical Applications, ser. A Wiley biomedical-health publication].

$$Sleep Efficiency = \frac{TotalSleepTime}{TotalMinutesinBed} = \frac{‖ SleepPeriod ‖ - WASO}{‖ SleepPeriod ‖ + Latency} \qquad ( 1.2 )$$

There is a massive market for better screening tools, as the majority of sleep conditions are left undiagnosed. This chapter surveys the ability of several statistical and data mining methods, to predict sleep efficiency proactively. It takes an in-depth look at analysing sensor time series with deep learning techniques for high fidelity prediction. It also introduces a novel deep leaning architecture called time-batched long short-term memory cells which builds off of the well-established long short-term memory cell spin-off from recurrent neural networks. This architecture shows a distinct improvement in its capability of handling the nature of wearable device data.

Diagnosing a condition requires insights beyond what standard deep learning methods are capable of. This chapter introduces a feature construction method via a human activity recognition algorithm. This algorithm, RAHAR (robust automated human activity recognition), provides a personalised activity labelling to a time series and a new level of automation that is not currently available in sleep research. Moreover, most human activity recognition algorithms aim to identify the type of behaviour. RAHAR is created to measure the exertion level of a behaviour.

Embodiments also provide recommendations for wearable device users to take action and improve their sleep quality. The methodology for extracting behavioural recipes through segmenting and clustering of the activity time series, in a retrospective manner is described. This novel way of building and evaluating a recommendation system allows for the rapid evolution of real-time sleep coaching.

### Approach

Introducing automation into the sleep science process is crucial for retrospective analysis. While retrospective analysis is useful in identifying whether or not the user endured a good or poor nights sleep, its conspicuous shortcoming is that it determines the quality of sleep after the fact. What is needed is a tool that can proactively predict the expected quality of sleep based on a user's activity. Recent systematic reviews have shown the relevance of physical activity to sleep, including sleep efficiency [M. A. Kredlow, M. C. Capozzoli, B. A. Hearon, A. W. Calkins, and M. W. Otto, “The effects of physical activity on sleep: a meta-analytic review,” Journal of behavioral medicine, vol. 38, no. 3, pp. 427-449, 2015; H. S. Driver and S. R. Taylor, “Exercise and sleep,” Sleep medicine reviews, vol. 4, no. 4, pp. 387-402, 2000; M. Chennaoui, P. J. Arnal, F. Sauvet, and D. Léger, “Sleep and exercise: a reciprocal issue?” Sleep medicine reviews, vol. 20, pp. 59-72, 2015]. Although the relationship between physical activity and sleep is not yet fully understood, it is thought to be a strong and complex correlation.

The importance of this approach is two-fold:

First, since some embodiments can be used in cases where sensory data during sleep is not available, the embodiments can be used in the early detection of potential low sleep efficiency. This is a common problem with consumer-grade wearable devices, as users might not wear them during the night (battery recharging, sensors embedded in smart jewellery, and so forth).

Second, the embodiments utilize advanced deep learning methods. Traditional prediction models applied to raw accelerometer data (e.g. logistic regression) suffer from at least 2 key limitations: (i) They are not robust enough to learn useful patterns from noisy raw accelerometer output. As a result, existing methods for classification and analysis of physical activity rely on extracting higher-level features that can be fed into prediction models [W. Wu, S. Dasgupta, E. E. Ramirez, C. Peterson, and G. J. Norman, “Classification accuracies of physical activities using smartphone motion sensors,” Journal of medical Internet research, vol. 14, no. 5, 2012]. This process often requires domain expertise and can be time consuming. (ii) Traditional methods do not exploit task labels for feature construction, and thus can be limited in their ability to learn task-specific features. Deep learning has the advantage that it is robust to raw noisy data, and can learn, automatically, higher level abstract features by passing raw input signals through non-linear hidden layers while also optimizing on the target prediction tasks. This characteristic was leveraged by building models using a range of deep learning methods on raw accelerometer data. This reduced the need for data preprocessing and feature space construction and simplified the overall workflow for clinical practice and sleep researchers.

### Automated Actigraphy

Automating the actigraphy process is the first step to improving the scalability of sleep disorder screening. A fully automated actigraphy process would examine the individual's activity signal and determine the Sleep Period, Sleep Onset, Awake Time, Time to Bed, Latency, WASO and Time to Active.

The raw actigraphy output from a clinically-validated device such as the Agtigraph GT3X+, includes raw accelerometer output as well as a signal over time of counts per minute. These counts are the sum of band-pass filtered accelerometer output. The counts vary based on the frequency and intensity of the signal [Actigraph gt3x+,” http://actigraphcorp.com/products-showcase/activity-monitors/actigraph-wgt3x-bt/, accessed: 2017-10-29]. As mentioned, the counts are measured on a per minute basis, but this can be altered to the desired epoch. Most consumer devices have the capability to collect at 30-100 Hz. FIG. 2 shows an example activity signal, i.e. counts per minute over time.

Once the activity signal is constructed, the sleep period can be identified. Traditional actigraphy generally includes a self-reported sleep diary that an individual fills in each night. The time to bed and the time to active is self-reported, and the time to sleep and time to awake is determined based on these boundaries. These records can be highly unreliable, especially in adolescents, so moving to an automated data-driven approach is again advantageous.

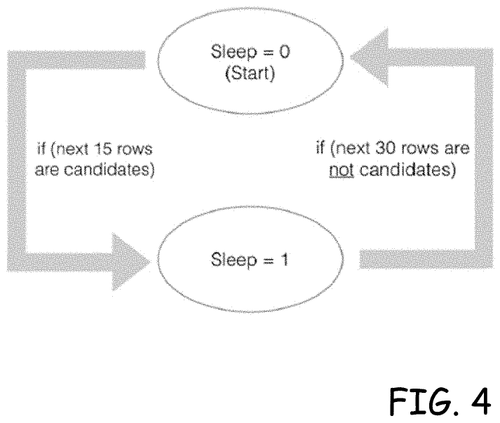

The first step is to run the entire raw accelerometer output through a state machine. FIG. 4 illustrates the state machine design. The accelerometer output is triaxial, on an {x,y,z} coordinate system. Rows refers to the aggregate accelerometer movement in an epoch of time, i.e. each epoch instance of {x,y,z}. Candidate Rows are rows with no triaxial movement, i.e. {x,y,z}={0,0,0}, indicating the device is stationary. Note that depending on the device, there are different methods for detecting whether the device is being worn or not. The methods mentioned here are applicable to all. By definition of the state machine, an individual is asleep (sleep onset time) after 15 consecutive candidate rows, or 15 continuous minutes of being perfectly stationary. Note that a sedentary individual would not be able to hold perfectly still for this period of time. After 30 consecutive non-candidate row, or 30 minutes of at least minor continuous activity, the user is considered awake. This marks the sleep awakenening time. The time period inbetween the sleep onset time and sleep awakenening time is considered the sleep period.

WASO, or wake after sleep onset, is measured by first identifying all moments of wakefulness within the sleep period boundaries as epochs with triaxial movement. If the wakefulness is continuous for over 5 minutes, it is aggregated into the WASO, otherwise it is discarded.

The Time to Bed and the Latency can be reverse engineered from the previous definitions with the use of human activity recognition.

### Deep Learning Methodology

In this section, the mathematics of the deep learning models (multi-layer perceptron, convolutional neural network, recurrent neural network, long short-term memory cells) is described, as well as the specifics on a novel architecture specifically for wearable data (time-batched long short-term memory cell).

Prediction Problem

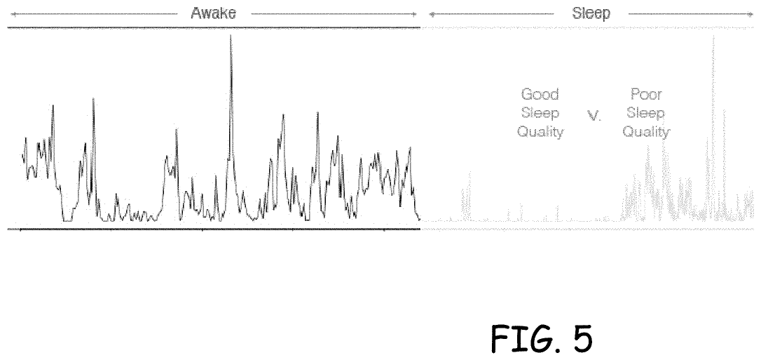

The input value to the prediction models is the activity signal, i.e. the counts per minute measured over time. The output is a binary classification of sleep quality as defined below. FIG. 5 illustrates the use of an individual's activity signal to predict sleep quality.

The general concept of sleep quality naturally falls into the binary classifications of “good sleep” and “poor sleep”. These are subjective measures, which is why many sleep science clinics use a patient questionnaire to determine the sleep quality experienced. In the move to an automated, data-driven approach, sleep efficiency is used as the measure of sleep quality. Note that while sleep efficiency is a strong measure of sleep quality, latency is a strong measure of perceived sleep quality.

In order to evaluate the effectiveness of the models, the sleep efficiency as measured from the automated actigraphy system is used. The sleep community considers a sleep efficiency below 0.85 as poor quality sleep, and sleep efficiency of above 0.85 as good quality sleep. Thus this threshold is used to divide the binary classifications.

### Deep Learning Modelling

Let xₜ [symbol] ^{D} be a vector representing a person's activity measured at time t. Given a series of such input vectors X=(x₁, . . . , x_{T}) representing a person's physical activity in an awake time period, the deep neural models first compute compressed representations with multiple levels of abstraction by passing the inputs through one or more non-linear hidden layers. The abstract representations of the raw activity measures are then used in the output layer of the neural network to predict the sleep quality. Formally, the output layer defines a Bernoulli distribution over the sleep quality y ∈ {good, poor}:

p(y|X, θ)=Ber(y|sig(w^{T}Ø(X)+b)) (2.1)

where refers to the sigmoid function, Ø(X) defines the transformations of the input X through non-linear hidden layers, w are the output layer weights and b is a bias term.

The models are trained by minimizing the cross-entropy between the predicted distributions ŷ_{nθ}=p(yₙ|Xₙ, θ) and the target distributions yₙ (i.e., the gold labels).

$$J ( θ ) = - {\sum }_{n} {y}_{n} log {\hat{y}}_{n θ} + ( 1 - {y}_{n} ) log ( 1 - {\hat{y}}_{n θ} ) \qquad ( 2.2 )$$

Minimizing cross-entropy is same as minimizing the negative log-likelihood (NLL) of the data (or maximizing log-likelihood). Unlike generalized linear models (e.g., logistic regression), the NLL of a deep neural model is a non-convex function of its parameters. Nevertheless, we can find a locally optimal maximum likelihood (ML) or maximum a posterior (MAP) estimate using gradient-based optimization methods. The main difference between the models, as we describe below, is how they compute the abstract representation Ø(X).

### Multi Layer Perceptrons

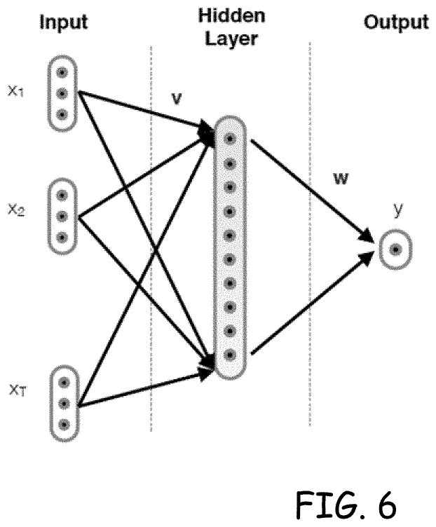

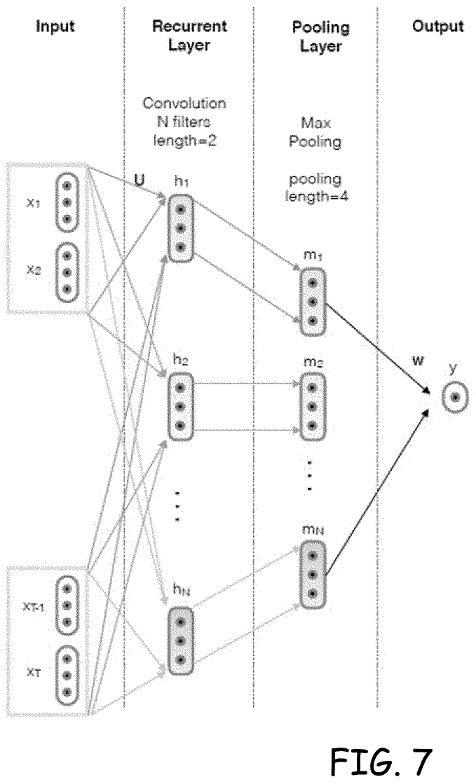

Multi-Layer Perceptrons (MLP), also known as Feed-forward Neural Networks, are the simplest models in the deep learning family. As shown in FIG. 6, the transformation of input Ø(X) in MLP is defined by one or more fully-connected hidden layers of the form

Ø(X)=f(Vₓ_{_{1:T}})=[f(v₁^{T}x_{1:T}), . . . , f(v_{N}^{T}x_{1:t})] (2.3)

where x_{1:T} is the concatenation of the input vectors x₁, . . . , x_{T}, V is the weight matrix from the inputs to the hidden units, f is a non-linear activation function (e.g., sig, tanh) applied element-wise, and N is the number of hidden units. MLP without any hidden layer (i.e., non-linear activations) boils down to a logistic regression (LR) or maximum entropy (MaxEnt) model. The hidden layers give MLP representational power to model complex dependencies between the input and the output.

By transforming a large and diverse set of raw activity measures into a more compressed abstract representation through its hidden layer, MLP can improve the prediction accuracy over linear models like LR.

### Convolutional Neural Networks

The fully-connected MLP described above has two main properties: (i) it composes each higher level feature from the entire input sequence, and (ii) it is time variant, meaning it uses separate (non-shared) weight parameter for each input dimension to predict the overall sleep quality. However, a person's sleep quality may be determined by his activity over certain (local) time periods in the awake time as opposed to the entire awake time, and this can be invariant to specific timings. For example, high intensity exercises or games over certain period of time can lead to good sleep, no matter when the activities are exactly performed in the awake time. Furthermore, each person has his own habit of activities, e.g., some run in the morning while others run in the afternoon. A fully-connected structure would require a lot of data to effectively learn these specific activity patterns, which is rarely the case in health domain. Convolutional Neural Networks (CNN) address these issues of a fully-connected MLP by having repetitive filters or kernels that are applied to local time slots to compose higher level abstract features. The weights for these filters are shared across time slots.

As shown in FIG. 7, the hidden layers in a CNN are formed by a sequence of convolution and pooling operations. A convolution operation involves applying a filter uᵢ ∈ [symbol] ^{L,D} to a window of L input vectors to produce a new feature hₜ

hₜ=f(uᵢ·x_{t:t+L-1}) (2.4)

where x_{t:t+L-1} denotes the concatenation of L input vectors and f is a non-linear activation function as defined before. We apply this filter to each window of L time steps in the sequence X to generate a feature map hᵢ=[h₁, . . . , h_{T+L-1}]. We repeat this process N times with N different filters to get N different feature maps h₁, . . . h_{N}. Note that we use a wide convolution rather than a narrow one, which ensures that the filters reach the entire sequence, including the boundary slots [N. Kalchbrenner, E. Grefenstette, and P. Blunsom, “A convolutional neural network for modelling sentences,” in Proceedings of the 52nd Annual Meeting of the Association for Computational Linguistics, June 2014]. This is done by performing zero-padding, where out-of-range (t<1 or t>T) slots are assumed to be zero.

After the convolution, we apply a max-pooling operation to each feature map

M=[μₚ(h₁), . . . , μₚ(h_{N})] (2.5)

where μₚ(hᵢ)=mᵢ refers to the max operation applied to each window of p features in the feature maph i. For p=2, this pooling gives the same number of features as in the feature map (because of the zero padding).

Intuitively, the filters compose activity measures in local time slots into higher-level representations in the feature maps, and max-pooling reduces the output dimensionality while keeping the most important aspects from each feature map. Since each convolution-pooling operation is performed independently, the features extracted become invariant in locations, i.e., when they occur in the awake time. This design of CNNs yields fewer parameters than its fully-connected counterpart, therefore, generalizes well for target prediction tasks.

### Recurrent Neural Networks

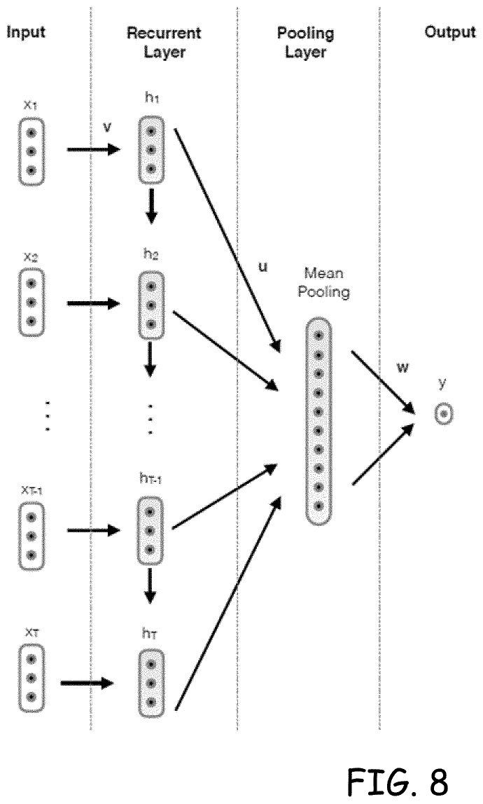

In CNN, features (or attributes) are considered in a bag-of-slots fashion disregarding the order information. The order in which the activities were performed in an awake time could be important. Recurrent Neural Networks (RNN) compose abstract features by processing activity measures in an awake time sequentially, at each time step combining the current input with the previous hidden state. More specifically, as depicted in FIG. 8, RNN computes the output of the hidden layer hₜ at time t from a nonlinear transformation of the current input xₜ and the output of the previous hidden layer hₜ₋₁. More formally,

hₜ=f(Uhₜ₋₁+Vxₜ) (2.6)

where f is a nonlinear activation function as before, and U and V are compositional weight matrices. RNNs create internal states by remembering previous hidden layer, which allows them to exhibit dynamic temporal behavior. We can interpret hₜ as an intermediate representation summarizing the past. The representation for the entire sequence can be obtained by performing a pooling operation (e.g., mean-pooling, max-pooling) over the sequence of hidden layers or simply by picking the last hidden layer h_{T}. In our experiments, we found mean-pooling to be more effective than other methods.

RNNs are generally trained with the backpropagation through time (BPTT) algorithm, where errors (i.e., gradients) are propagated back through the edges over time. One common issue with BPTT is that as the errors get propagated, they may soon become very small or very large that can lead to undesired values in weight matrices, causing the training to fail. This is known as the vanishing and the exploding gradients problem [Y. Bengio, P. Simard, and P. Frasconi, “Learning long-term dependencies with gradient descent is difficult,” IEEE Transactions on Neural Networks, vol. 5, no. 2, pp. 157-166, 1994]. One simple way to overcome this issue is to use a truncated BPTT [T. Mikolov, Statistical Language Models based on Neural Networks. plus 0.5em minus 0.4emPhD thesis, Brno University of Technology, 2012] for restricting the backpropagation to only few steps like 4 or 5. However, this solution limits the simple RNN to capture long-range dependencies. Below, an elegant RNN architecture in accordance with one embodiment is described that addresses this problem.

### Long Short-Term Memory Cell Recurrent Neural Networks

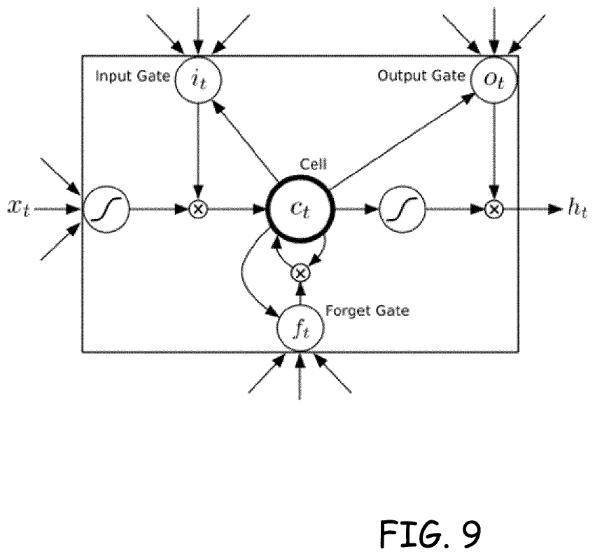

Long Short-Term Memory or LSTM [S. Hochreiter and J. Schmidhuber, “Long short-term memory,” Neural computation, vol. 9, no. 8, pp. 1735-1780, 1997] is specifically designed to capture long range dependencies in RNNs. The recurrent layer in a standard LSTM is constituted with special units called memory blocks (FIGS. 8 and 9). A memory block is composed of four elements: (i) a memory cell c (a neuron) with a self-connection, (ii) an input gate i to control the flow of input signal into the neuron, (iii) an output gate o to control the effect of the neuron activation on other neurons, and (iv) a forget gate f to allow the neuron to adaptively reset its current state through the self-connection. The following sequence of equations describe how the memory blocks are updated at every time step t:

iₜ=sigh(Uᵢhₜ₋₁+Vᵢxₜ+b₁) (2.7)

fₜ=sigh(U_{f}hₜ₋₁+V_{f}xₜ+b_{f}) (2.8)

cₜ=iₜ⊙ tanh(U_{c}hₜ₋₁+V_{c}xₜ)+fₜØcₜ₋₁ (2.9)

oₜ=sigh(Uₒhₜ₋₁+Vₒxₜ+bₒ) (2.10)

hₜ=oₜØtanh(cₜ) (2.11)

where Uₖ and Vₖ are the weight matrices between two consecutive hidden layers, and between the input and the hidden layers, respectively, which are associated with gate k (input, output, forget and cell); and bk is the corresponding bias vector. The symbols sigh and tanh denote hard sigmoid and hard tan, respectively, and the symbol Ø denotes an element-wise product of two vectors. LSTM by means of its specifically designed gates (as opposed to simple RNNs) is capable of capturing long range dependencies.

### Time-batched Long Short-Term Memory Cell Recurrent Neural Networks

The naive way to apply RNNs to activity records is to consider the accelerometer output aggregated at every minute in an awake time as a separate time step. In our data, each time step contains one measure (i.e. the vertical axis). This results in very lengthy sequences with only one feature at each time step. RNNs applied to this setting suffer from two problems: (i) because of the low dimensional input at each time step, RNNs become ineffective in composing features and fail to capture the sequential dependencies, and (ii) because of long sequences, the gradients (errors) from the final time step vanish before they reach to earlier parts of the network, causing the training with BPTT to fail even with LSTM cells.

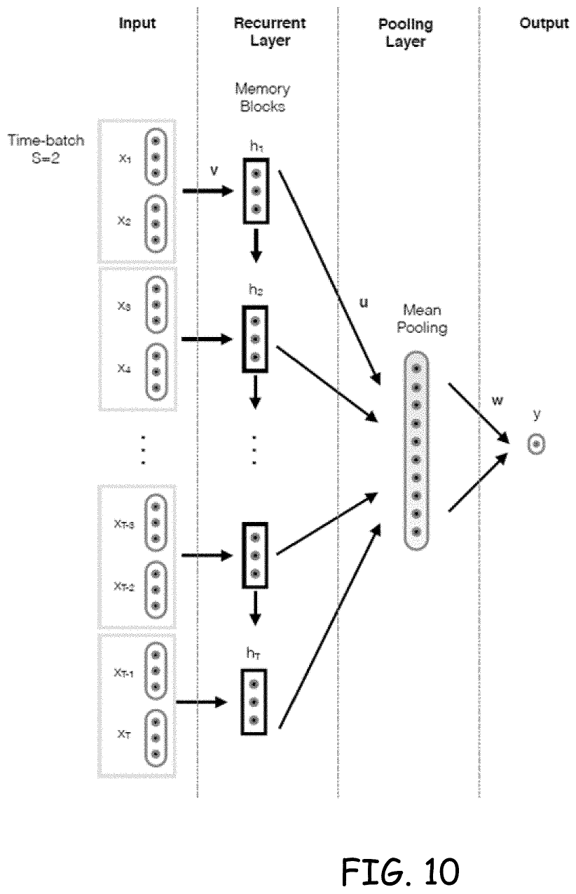

To circumvent this problem, we construct batches of time steps by merging accelerometer measures over S time steps. In other words, each time step in the batch setting generates an input vector of S×D elements. To make all input vectors equal-sized, we use zero-padding for the last time step (if needed). Note that setting S=1 gives the original sequences. FIG. 10 provides an example of a Time-Batched Long Short-Term Memory Cell Recurrent Neural Network Architecture.

### Experimental Design

In this section, the experimental design, details on the data used, and experimental results are described.

Data Collection

The data used in one experiment was collected as part of a research study to examine the impact of sleep on health and performance in adolescents by Weil Cornell Medical College-Qatar. Two international high schools were selected for cohort development. The participants consisted of 92 male and female adolescents from ages 10 to 17.

Student volunteers were provided with an actigraph accelerometer, ActiGraph GT3X+1, to wear on their non-dominant wrist continuously throughout the one-week observational trial. The device was water resistant and fully charged so the device would not need to be removed for any reason (i.e. even when sleeping and showering). Deidentified data collected in the study were used in this analysis. The wearable device sampled the user's sleep-wake activity at 30-100 Hertz. Currently sleep experts use this device in conjunction with the accompanying software, ActiLife [Actigraph gt3x+,” http://actigraphcorp.com/products-showcase/activity-monitors/actigraph-wgt3x-bt/, accessed: 2017-10-29], to evaluate an individual's sleep period. We evaluate our results side-by-side with ActiLifes results.

### 2.4.2 Data Partitioning

To train the models without over-fitting and test their performance afterward, a random partitioning of the dataset was created. Each time series was assigned to a partition randomly while maintaining an even class distribution of the target variable, sleep quality. The data were split with a 70%-15%-15% ratio for training, testing, and validation sets, respectively. All reported results are based on model predictions on the test set.

### Data Staging

The input of the models were time series vectors, X=(x₁ . . . , x_{T}), representing the physical activity of a person's awake time. Each vector corresponded to a continuous period of awake time, and so for each individual, there might be multiple such vectors over the 7 days. Each x_{T} represented the triaxial value of the vertical axis at time t.

The output of the model was a binary classification decision between good and poor sleep quality based on the sleep efficiency equation (%). These classifications were determined by using automated actigraphy to evaluate the sleep quality retrospectively, and were used as the ground truth. Good quality sleep corresponds to a sleep efficiency above 0.85, and poor quality sleep corresponds to an efficiency below or equal to 0.85. In addition to the binary decision, the model also gave its confidence (a score between 0.0 and 1.0) in that decision.

### Model Training

To be able to predict, the models were first trained on the training dataset, using an online training algorithm, RMSprop [T. Tieleman and G. Hinton, “Lecture 6.5-rmsprop: Divide the gradient by a running average of its recent magnitude,” COURSERA: Neural networks for machine learning, vol. 4, no. 2, pp. 26-31, 2012], which relied on a number of preset parameters:

Dropout ratio: The ratio of hidden units to turn off in each mini-batch training.

Mini-batch size: The number of training instances to consider at one time.

Learning rate: The rate at which the parameters are updated.

Max epoch: The maximum number of iterations over the training set.

The training algorithm minimizes the cross-entropy between the predicted distribution and the actual (ground truth) target labels. To avoid over-fitting, we used early stopping based on the models performance on the validation set. In particular, the model was evaluated after every epoch on the validation set and stopped when its accuracy went down. To reduce the cross-entropy between the predicted distributions and the target distributions, RMSprop was used setting the maximum number of epochs to 50.

Rectified linear units (ReLU) were used for the activation functions (f). Dropout [N. Srivastava, G. Hinton, A. Krizhevsky, I. Sutskever, and R. Salakhutdinov, “Dropout: A simple way to prevent neural networks from overfitting,” Journal of Machine Learning Research, vol. 15, pp. 1929-1958, 2014] of hidden units was also used to avoid overfitting. Regularization on weights such as l₁ and l₂ did not improve the results. We experimented with DR ∈ {0.0, 0.1, 0.2, 0.3, 0.4, 0.5} dropout rates and M B ∈ {5, 10, 15, 20} mini batch sizes. For MLP, we experimented with one hidden layer containing N ∈ {2, 3, 5, 10, 15, 20} units. For CNN, we experimented with N ∈ {25, 50, 75, 100, 125, 150} number of filters with filter lengths F L ∈ {2, 3, 4, 5} and pooling lengths P L ∈ {2, 3, 4, 5}. For simple and LSTM RNNs, we experimented with N ∈ {25, 50, 75, 85, 100} hidden units in the recurrent layer, and S ∈ {25, 50, 75, 100} time-slots for constructing batches in the time-batched LSTM. Since the size of the training data is small, the network weights are initialized with zero to start the training with the simplest model. Optimal model configurations for each setting is summarized in table 2.1 and the following subsections.

### Logistic Regression

As a baseline model, we used logistic regression (LR) to predict the sleep quality. LR is a generalized linear classification model that does not have any hidden layers. For the LR, the raw input signals, X, are directly fed into the output layer for prediction without any nonlinear hidden layer transformations. The optimal setting for logistic regression (LR) was with a mini-batch size of 5 and a dropout ratio of 0.5.

TABLE 2.1
Optimal Model Configurations

| | Model | | | | | | |
|---|---|---|---|---|---|---|---|
| | | | | | | | TB- |
| | MLP* | LR | MLP | CNN | RNN | LSTM | LSTM |
| Dropout Ratio | 0.3 | 0.5 | 0.1 | 0.0 | 0.1 | 0.5 | 0.5 |
| Mini-Batch Size | 5 | 5 | 20 | 5 | 5 | 5 | 5 |
| Hidden Layer | 15 | — | 15 | — | 75 | 100 | 100 |
| Size | | | | | | | |
| Time Batch Size | — | — | — | — | — | — | 50 |
| Number of Filters | — | — | — | 25 | — | — | — |
| Filter Length | — | — | — | 5 | — | — | — |
| Pool Length | — | — | — | 4 | — | — | — |

### Multi-Layer Perceptron

MLPs, also known as feed-forward neural networks, are the simplest models in the deep learning family. They have one or more hidden layers. In fact, MLP without any hidden layers is equivalent to logistic regression. In MLP, all the units of a hidden layer are fully connected to the units in the previous layer. The best parameter configuration for MLP was with a mini-batch size of 20, a dropout ratio of 0.1, and a hidden layer size of 15.

### Convolutional Neural Network

CNNs are a more complex type of deep learning method that includes repetitive filters or kernels applied to local time slots, thereby composing a high level of abstract features. This operation is called convolution. After convolution, a max-pooling operation is performed to select the most significant abstract features. This design of CNNs yields fewer parameters than its fully connected counterpart (MLP), and therefore generalizes well for target prediction tasks. For its best configuration, we used 25 hidden nodes, filter length of 5 and pooling length of 4, 5 mini-batch size, and 0.0 dropout ratio.

### Recurrent Neural Network

RNNs compose abstract features by processing activity measures in an awake time sequentially, at each time step combining the current input with the previous hidden state. RNNs create internal states by remembering the previous hidden layer, which allows them to exhibit dynamic temporal behavior. These features make RNNs a good deep learning method for temporal series. RNNs performed best with a mini-batch size of 5, a dropout ratio of 0.1 and a hidden layer size of 75.

### Long Short-Term Memory Cell Recurrent Neural Network

A subtype of RNN, LSTM uses specifically designed memory blocks as units in the recurrent layer to capture longer-range dependencies. The optimal configuration values for LSTM were a mini batch size of 5, dropout ratio of 0.5, and hidden layer size of 100.

### Time-Batched Long Short-Term Memory Cell Recurrent Neural Network

To further improve our implementation of LSTM, we constructed batches of time steps by merging accelerometer measures over time steps. We referred to this version of the model as TB-LSTM. The optimal configuration values for TB-LSTM were mini-batch size of 5, dropout ratio of 0.5, and hidden layer size of 100.

### Evaluation

For the evaluation of the performance of the different models, several well-known metrics such as accuracy, precision, recall, F1-score, and area under the receiver operating characteristic (ROC) curve (AUC), are used. These metrics are commonly used in data mining and clinical decision support systems.

### Accuracy

It is computed as the proportion of correct predictions, both positive and negative (sum of true positives and true negatives divided by the number of all instances in the dataset).

### Precision

It is the fraction of the number of true positive predictions to the number of all positive predictions (i.e., true positives divided by the sum of true positives and false positives). In our case, precision described what percentage of the time the model predicted good-quality sleep correctly. Note that precision is also known as positive predictive value.

### Specificity

It is the fraction of the number of true negative predictions to the actual number of negative instances in the dataset (i.e. true negatives divided by the sum of true negatives and false positives). In our case, specificity referred to the percentage of the correctly predicted poor-quality sleep to the total number of poor-quality sleep instances in the dataset. Note that specificity is also known as true negative rate.

### Recall or Sensitivity

It is the fraction of the number of true positive predictions to the actual number of positive instances in the dataset (i.e. true positives divided by the sum of true positives and false negatives). In our case, recall referred to the percentage of the correctly predicted good-quality sleep to the total number of good-quality sleep instances in the dataset. Note that recall is also known as true positive rate or sensitivity.

### F1-Score

There is usually an inverse relationship between precision and recall. That is, it is possible to increase the precision at the cost of decreasing the recall, or vice versa. Therefore, it is more useful to combine them into a single measure such as F1 score, which computes the harmonic mean of precision and recall.

### Area Under the ROC Curve

It represents the probability that a classifier will rank a randomly chosen positive instance higher than a randomly chosen negative instance. Hence, AUC defines an effective and combined measure of sensitivity and specificity (which are often inversely related, just like precision and recall) for assessing inherent validity of a classifier.

### Results

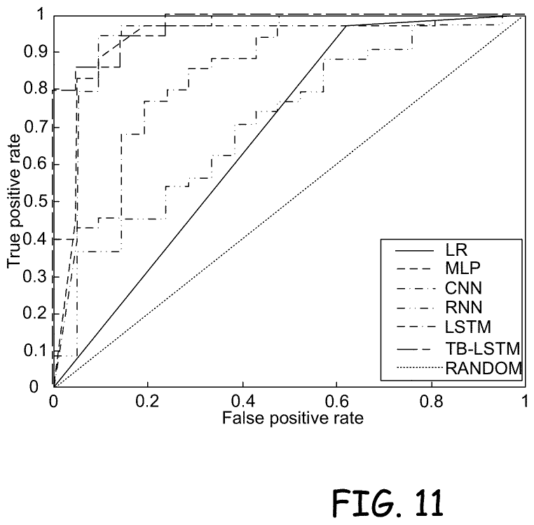

As shown in table 2.2 and FIG. 11, the performance of the logistic regression in the metrics previously explained, performed worse than the models based on deep learning. Only the simple RNN performed worse than logistic regression in both F1-score (harmonic mean of precision and recall) and accuracy.

Also shown in table 2.2, the AUC of the logistic regression model was low. The AUC value for logistic-regression was 0.6463, which was close to 0.5 (equivalent to a random prediction). This showed the limitation of classical models in analyzing raw accelerometry.

In contrast, all the AUC values for the deep learning models were better with a range from 0.7143 to 0.9714, TB-LSTM being the best performer and RNN, the worst. Time-batched LSTM, CNN, and MLP performed the best with AUC scores showing an improvement over LR by 50%, 46%, and 46%, respectively.

TABLE 2.2
Deep Learning Results on Raw Accelerometer Data

| | AUC | F1-Score | Precision | Recall | Accuracy |
|---|---|---|---|---|---|
| LR | 0.6463 | 0.8193 | 0.7083 | 0.9714 | 0.7321 |
| MLP | 0.9449 | 0.9118 | 0.9394 | 0.8857 | 0.8929 |
| CNN | 0.9456 | 0.9444 | 0.9189 | 0.9714 | 0.9286 |
| RNN | 0.7143 | 0.7711 | 0.6667 | 0.9143 | 0.6607 |
| LSTM | 0.8531 | 0.8500 | 0.7556 | 0.9714 | 0.7857 |
| TB-LSTM | 0.9714 | 0.9211 | 0.8537 | 1.0000 | 0.8929 |

### Comparison Between Deep Learning Models

Upon comparing the deep learning neural network models, it is apparent that CNN yielded slight improvement over MLP in AUC (0.07% absolute), but more in F1 (3.57%) and accuracy (4.00%).

These improvements could be attributed to the time-invariant convolution-pooling operations of the CNN model to pick key local patterns, which generalized well for small training data. The F1-score improved by 12% for time-batched LSTM, by 15% for CNN, and by 11% for MLP. The accuracy improved by 22% for time-batched LSTM, by 27% for CNN, and by 22% for MLP.

A comparison of the RNN models revealed that LSTM outperformed simple RNN by a wide margin; 19%, 10%, and 19% in AUC, F1, and accuracy, respectively. These gains over simple RNNs could be attributed to the specially designed gates of LSTMs that could capture long-range dependencies between physical activities in the sequences.

However, this is not surprising. Both simple and LSTM RNNs operate on sequences, where each time step comprises only one activity value. This often results in very long sequences. As mentioned earlier, in this setting RNNs cannot compose higher-level features effectively because of low-dimensional input at each time step, and also suffer from vanishing gradient problems due to lengthy sequences.

The solution to surmount this problem was to use a time-batched input to LSTM. When comparing the results of the novel time-batched LSTM (TB-LSTM) with those of MLP and CNN, the TB-LSTM outperformed both MLP and CNN in AUC by 3%; in fact, it had the highest AUC score. It achieved better F1 score than MLP (1%), but worse than CNN (2%). When observing their precision and recall values, TB-LSTM had a very high recall but lower precision, which meant that it tended to predict more good quality sleep than the gold standard. For the same reason, its accuracy was also lower than that of CNN.

### Discussion

### Principal Findings

This study focused on the prediction of poor versus good sleep efficiency. It is a simple, but important, problem as sleep efficiency has been found to be a crucial sleep parameter with important health consequences [J. Park, M. M. Martinez, M. H. Berlinger, A. Ringrose, D. J. Clifton, S. E. McKinney, and G. Amit, “Wristband health tracker,” Feb. 9, 2016, U.S. Pat. D749,002; R. Budhiraja, R. Thomas, M. Kim, and S. Redline, “The role of big data in the management of sleep-disordered breathing,” Sleep medicine clinics, vol. 11, no. 2, pp. 241-255, 2016; R. J. Cole, D. F. Kripke, W. Gruen, D. J. Mullaney, and J. C. Gillin, “Automatic sleep/wake identification from wrist activity,” Sleep, vol. 15, no. 5, pp. 461-469, 1992]. Furthermore, the prediction did the estimate the overall sleep efficiency but rather the differentiation between two classes (poor versus good sleep efficiency). This classification is consequently not an indicator of sleep patterns, but the prediction of a sleep quality parameter that might indicate a potential sleep problem.

As in prediction or diagnostic problems, the results need to be discussed in terms of sensitivity and specificity (see table 2.3). The deep learning methods of CNN and TB-LSTM were the best performers overall. Their sensitivity (0.97 and 1, respectively) showed that these models were able to detect nearly all the cases of good-quality sleep, meaning that in a tool for screening potential sleep problems these models will be able to detect easily people with normal sleep quality. Often high sensitivity comes at the price of low specificity (i.e., failing to identify negative cases, or true negative error). This was the case of logistic regression, which had a high sensitivity but a specificity of 0.3, meaning that in such models many poor sleeps would have been wrongly classified as good sleep. This is very important, since misidentifying poor sleep cases can lead to under-diagnosis of problematic sleep.

The sensitivity (also known as recall) and specificity of each of the models are reported in table 2.3. The high sensitivity values of each of the models indicate that deep learning has a strong capability of correctly identifying individuals with good sleep patterns from their preceding awake activity. The specificity is high for TB-LSTM, MLP, and CNN, indicating that these models were also able to successfully distinguish those with poor sleep patterns.

TABLE 2.3
The Sensitivity and Specificity of the Deep Learning Models

| | Sensitivity | Specificity |
|---|---|---|
| LR | 0.9714 | 0.3333 |
| MLP | 0.8857 | 0.9048 |
| CNN | 0.9714 | 0.8571 |
| RNN | 0.9143 | 0.2381 |
| LSTM | 0.9714 | 0.4762 |
| TB-LSTM | 1.0000 | 0.7143 |

Impact on Sleep Science and eHealth

Millions of consumers are purchasing wearable devices that incorporate activity sensors. This burst of human activity data is a great opportunity for health research, but to achieve this paradigm shift, it is necessary to develop new algorithms and tools to analyses this type of data. Furthermore, sleep insufficiency is highly prevalent in contemporary society, and has been shown to influence energy balance by altering metabolic hormone regulation. Consequently, health researchers are exploring the impact of sleep and physical activity on many health conditions. A major bottleneck for this research is that current approaches for studying actigraphy data require intensive manual work by human experts. Moreover, huge datasets of actigraphy data are emerging from health research, including the study of sleep disorder patients, healthy populations, and epidemiological studies.

As explained in the results, this research supports the feasibility of using physical activity data to predict the quality of sleep in terms of sleep efficiency. Improved algorithms, such as the ones presented in this study, can lead to a paradigm shift in the study of lifestyle behaviors such as sleep and physical activity, just as electrocardiography became crucial for cardiology and clinical research.

This work provides an early example of how advanced deep learning methods can be used to infer new insights from raw actigraphy data. The results illustrate that deep learning performed better than classical methods in terms of learning useful patterns from raw accelerometer data for the task of sleep quality prediction. Since deep learning models compute hidden features from raw input signals while optimizing on the actual sleep quality, this process yields a more robust solution. Furthermore, the good results of deep learning show that raw accelerometer data had more signal regarding sleep quality, which traditional models such as logistic regression are not able to capture.

The deep learning algorithms predicted a parameter regarding the quality of sleep solely relying on the physical activity during the awake time. The advantage of this approach is that eventually the same technique can be used to predict sleep quality with data from smart watches and other wearable devices that are not necessarily used during sleep. Therefore, our models can eventually be used within eHealth applications that do not require wearing a sensor during sleep. This is of special interest for the development of smart watch health applications [T.-C. Lu, C.-M. Fu, M. H.-M. Ma, C.-C. Fang, and A. M. Turner, “Healthcare applications of smart watches: a systematic review,” Applied clinical informatics, vol. 7, no. 3, p. 850, 2016], as they might require frequent battery charging.

### Conclusion

The embodiments above provide two significant improvements over the prior art. First, the embodiments automate actigraphy analysis. This system eliminates the sleep clinic bottle neck and has the potential to greatly reduce the number of undiagnosed sleep conditions, by alerting users of serious problems. It also allows the consumer with an interest in optimizing their sleep, to evaluate their activity with their own wearable device.

The second contribution was the ability to make a proactive sleep quality prediction based on an individual's daily activity. The results show the feasibility of deep learning in predicting sleep efficiency using wearable data from awake periods. Our architecture showed an ability to interpret the specific low-dimensional and longitudinal nature of wearable device data. This is of particular importance because deep learning eliminates the need for data preprocessing and simplifies the overall workflow in clinical care and sleep research. The feasibility of this approach can lead to new applications in sleep science and also to the development of more complex eHealth sleep applications for both professionals and patients. These models can also be integrated in the broader context of quantified self.

### Diagnosis

The biggest shortcoming of the deep learning tools introduced above is the lack of transparency. Deep learning tools are ‘black boxes’ in that do not explain why a decision or prediction was made. Although there are many researchers working on ‘unboxing’ the algorithmically determined rules, this research is still preliminary. “Black box” results are particularly problematic for some of the disciplines that artificial intelligence and deep learning are expected to revolutionize. These tasks (such as high-stake trading decisions, loan approval, autonomous driving, etc.) can lead to very serious consequences. In fact, in 2018 the European Union may require companies to justify decisions made my autonomous systems [W. Knight, “The dark secret at the heart of al,” TECHNOLOGY REVIEW, vol. 120, no. 3, pp. 54-61, 2017].

In the case of sleep screening, determining whether or not a sleep problem is present, is the first step. The second step requires diagnosing the condition. While the deep learning methods provided a high-fidelity prediction model for sleep quality, they provided no insights or information into why that sleep quality was occurring. The models automatically learnt hidden feature representations from the raw accelerometer input. In this chapter, a method for creating interpretable feature representations is presented. The goal of these representations is threefold:

To improve the prediction ability of downstream analysis using traditional data mining methods, such as support vector machines and decision trees. These methods struggle to perform on the raw accelerometer data.

To provide insights and interpretability into the rationale behind the predictions. Traditional data mining modelling will allow for this.

To advance automated actigraphy to the next level, and provide personalized measurements of an individual's activity.

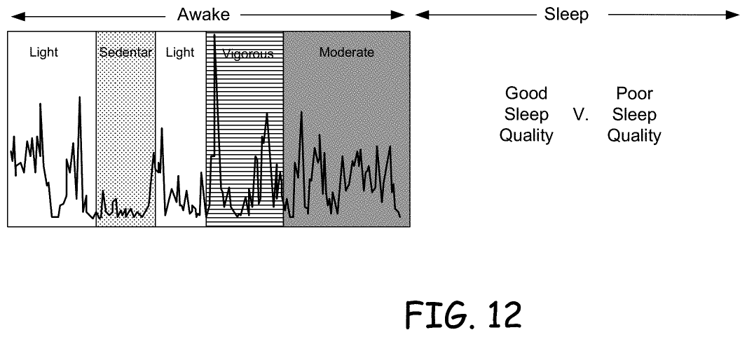

Currently, the sleep science process uses polysomnography, home sleep tests and actigraphy to diagnose sleep conditions. All of these methods require in clinic visits, and rely on clinical experts to manually evaluate the data. The automated actigraphy process described above identifies the key actigraphy milestones and replaces this cumbersome process. However, this process can be taken one step further by the incorporation of human activity recognition algorithms. FIG. 12 illustrates the usage of human activity recognition features for sleep quality prediction.

### 3.1 Approach

Human Activity Recognition (HAR) is the understanding of human behaviour from data captured by pervasive sensors, such as cameras or wearable devices. It is a powerful tool in medical application areas, where consistent and continuous patient monitoring can be insightful. Wearable devices provide an unobtrusive platform for such monitoring, and due to their increasing market penetration, feel intrinsic to the user. This daily integration into a user's life is crucial for increasing the understanding of overall human health and wellbeing.

Wearables, such as actigraph accelerometers, generate a continuous time series of a person's daily physical exertion and rest. This ubiquitous monitoring presents substantial amounts of data, which can (i) provide new insights by enriching the feature set in health studies, and (ii) enhance the personalization and effectiveness of health, wellness, and fitness applications. By decomposing an accelerometers time series into distinctive activity modes or actions, a comprehensive understanding of an individual's daily physical activity can be inferred. The advantages of longitudinal data are however complemented by the potential of noise in data collection from an uncontrolled environment. Therefore, the data sensitivity calls for robust automated evaluation procedures.

In one embodiment, a robust automated human activity recognition (RAHAR) algorithm [A. Sathyanarayana, F. Ofli, L. Fernandez-Luque, J. Srivastava, A. Elmagarmid, T. Arora, and S. Taheri, “Robust automated human activity recognition and its application to sleep research,” in Data Mining Workshops (ICDMW), 2016 IEEE 16th International Conference on. plus 0.5em minus 0.4emIEEE, 2016, pp. 495-502] is presented. The algorithm is tested in the application area of sleep science by providing a novel framework for evaluating sleep quality and examining the correlation between the aforementioned and an individual's physical activity. Even though the performance of the proposed HAR algorithm is evaluated on sleep analysis, RAHAR can be employed in other research areas such as obesity, diabetes, and cardiac diseases.

### Methodology

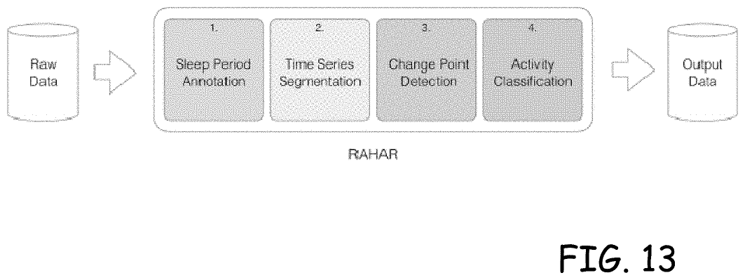

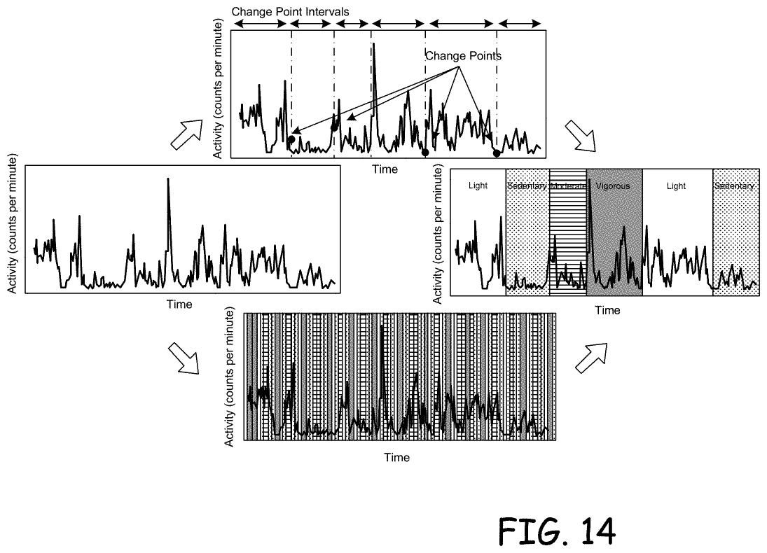

The methodology for RAHAR is shown algorithmically in FIG. 13. We elaborate on the details of the algorithm in this section. FIG. 14 provides a high-level overview of key steps in the RAHAR pipeline. The right most graphic illustrates the robust and automated human activity recognition labels on the time series.

### Sleep Period Annotation

The sleep period annotation phase builds on top of automated actigraphy. However, since RAHAR can be used as a standalone tool for human activity recognition in any domain, it is not assumed that automated actigraphy is completed, and we start with the raw, non-annotated activity signal. The accelerometer output is put through the state machine in FIG. 4. The details of this process are in section 2.2.

The key outputs from the state machine are the sleep onset time and sleep awakening time. These terms border the sleep period, but in reverse, also border the awake time of an individual. As shown in FIG. 12, RAHAR identifies exertion levels of an individual through their awake time, so identifying this time period is critical.

### Time Series Segmentation

Since wearable device data is collected continuously, the time series can carry on over multiple awake and sleep periods. For this reason, the time series is divided into multiple awake-time-sleep-time pairs, by segmenting the time series at every awake time. Since RAHAR can be used as a diagnostics tool for sleep quality prediction, the human activity recognition determinations in the preceding awake time, are used to predict the following sleep period. This process is described in further detail in the evaluation section.

### Change Point Detection

One of the key components of RAHAR is its personalization. RAHAR does not create arbitrary divisions in activity (e.g. cut-offs every hour), or create population level generalizations, (e.g., count per minutes thresholds). Rather it uses, change point detection to find localized distributional changes within the local time series that indicate a change in behaviour. This is done for each individual time series using a method called hierarchical divisive estimation.

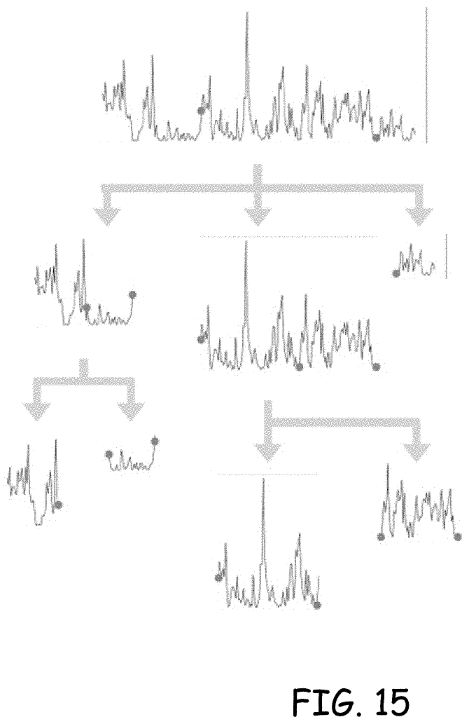

Hierarchical divisive estimation can be broken down into three fundamental steps: (i) measure the difference in the distribution, (ii) estimate the location of the change points hierarchically, and (ii) test the significance of the change point. The mathematical details of how an individual change point is found are described below. For further details please refer to the original paper, [N. James and D. Matteson, “ecp: An r package for nonparametric multiple change point analysis of multivariate data,” Journal of Statistical Software, vol. 62, no. 1, pp. 1-25, 2015]. FIG. 15 illustrates how the change points are identified hierarchically. Upon each division in the time series, points are located where the distribution differs. These points are then tested for statistical significance. If the point is statistically significant, it is considered a change point, and the time series is divided. This process continues iteratively in a hierarchical manner, until no further statistically significant change points can be identified.

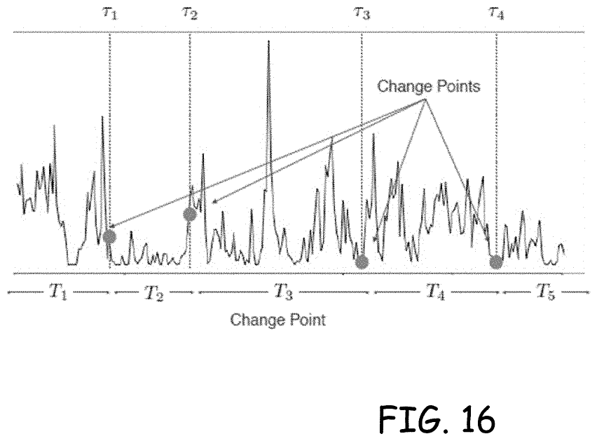

Let x₁, x₂, . . . , x_{T} ∈ R be the activity signal of counts per minute over time, as seen in FIG. 16. Define Yₙ={Yᵢ: i=1, 2, . . . , n} and Zₘ=Zⱼ: j=1, 2, . . . , m. This gives the sample divergence measure as:

$$\hat{ɛ} ( {Y}_{n} , {Z}_{m} ( k ) ; α ) = \frac{2}{mn} {\sum }_{i = 1}^{n} {\sum }_{j = 1}^{m} {\vert {Y}_{i} - {Z}_{j} \vert}^{α} - {( \begin{matrix}n \\ 2\end{matrix} )}^{- 1} {\sum }_{1 \leq i < k \leq n} {\vert {Y}_{i} - {Y}_{k} \vert}^{α} - {( \begin{matrix}m \\ 2\end{matrix} )}^{- 1} {\sum }_{1 \leq j < k \leq m} {\vert {Z}_{j} - {Z}_{k} \vert}^{α} \qquad ( 3.1 )$$

where the scaled empirical divergence is:

$$\hat{Q} ( {Y}_{τ} , {Z}_{τ} ( k ) ; α ) = \frac{mn}{m + n} \hat{ɛ} ( {Y}_{τ} , {Z}_{τ} ( k ) ; α ) \qquad ( 3.2 )$$

Defining, Y_{τ}=x₁, x₂, . . . , x_{τ} and Z_{τ}(k)=x_{τ+1}, x_{τ+2}, . . . , xₖ where 1≤τ<k≤T. the change point is then:

$$( \hat{τ} , \hat{k} ) = \begin{matrix}argmax \\ ( τ , k )\end{matrix} \hat{Q} ( {Y}_{τ} , {Z}_{τ} ( k ) ; α ) \qquad ( 3.3 )$$

As mentioned, the segment is tested to find a point that could divide the time series such that the division would create 2 vectors with maximum divergence. For the purposes of implementation, a maximum number of random permutations of 99 was selected.

The purpose of identifying the change points is to identify the key moments in an activity time series, where there is a quantitative change in behaviour. FIG. 16 shows how the activity time series would be segmented. Each change point makes the boundaries of a change-point-interval.

### Activity Classification

The activity signal from the accelerometer data contains post-filtered counts for each of the {x,y,z} axes. These counts quantify the frequency and intensity of the user activity. Using Troianos cut point scale [R. P. Troiano, D. Berrigan, K. W. Dodd, L. C. Masse, T. Tilert, M. McDowell et al., “Physical activity in the united states measured by accelerometer,” Medicine and science in sports and exercise, vol. 40, no. 1, pp. 181-188, 2008], the age of a user, and their accelerometer triaxial count, each epoch in the time series can be labeled with an intensity level: sedentary, light, moderate, or vigorous activity.

In this case, each epoch is one minute in length. Interpreting an individual's behaviour at a minute-by-minute level provides an unnecessary granularity. Moreover, it is highly subject to noise. Aggregating the cut point activity intensity labels, over the change point intervals, smooths the signal and provides a more robust learning representation. To determine the exertion level of each change point interval, RAHAR calculates the statistical mode of the cut point labels over each epoch.

Not only does this process personalize the exertion level interpretation, but it automates the activity labelling, eliminating the need for cumbersome annotation by sleep experts, and enabling the widespread evaluation required in large clinical trials.

### Experimental Design

RAHAR is fundamentally a feature extraction algorithm for human activity recognition, in the context of quantifying daily physical activity exertion levels of individuals. Although RAHAR can be used in other domains, it is tested here as a tool for sleep science diagnosis. As a result, the evaluation of RAHAR should include an assessment of its output, used as input into a prediction model for sleep quality. As opposed to the algorithms used above, using RAHAR, will provide insights into the behaviour of an individual.

The best evaluation, is to test the quality of activity recognition by RAHAR as compared to the current process, i.e. an expert using actigraphy software to label the exertion levels. There is no ground truth on human activity in this context, in other words there is no way of knowing the reality of what an individual exerted. Instead, the objective is to evaluate which approach leads to better higher-fidelity models for predicting sleep quality.

Four models were selected for evaluating the performance of RAHAR against the performance of an expert-annotated time series: logistic regression, support vector machines with radial basis function kernel, random forest, and adaboost.

Logistic Regression (LogR): This model was chosen because it is an easily interpretable binary classifier. It is also relatively robust to noise, which as explained earlier is a complication on data collected in an uncontrolled environment. [Note: Even though logistic regression (LogR) is included is important to note that LogR model failed to stratify the dataset successfully for the state-of-the-art baseline approach, and predicted all cases to be in a single class. Therefore, the LogR score of RAHAR is excluded from analysis whenever corresponding LogR score of the state-of-the-art baseline approach were not available.]

Support Vector Machine (SVM): This model was selected because it, also, is a binary classifier. using a radial basis function kernel, it differs from logistic regression in that it does not linearly divide the data.

Random Forest (RF): This model was tested as an alternative because of its easy straightforward interpretation, which is particularly relevant in the healthcare or consumer domains. It also is not restricted to linearly dividing the data.

Adaboost (Ada): Lastly, Adaboost was tested because it is less prone to overfitting than random forest.

### Results

Table 3.1 and 3.2 disclose the results of using RAHAR on the data with the different prediction models. ‘SE+AL’ refers to a sleep expert using the ActiLife software, as opposed to the automated process of RAHAR.

TABLE 3.1
RAHAR Prediction Evaluation Metrics

| | AU-ROC | | F1 Score | | Accuracy | | Precision | |
|---|---|---|---|---|---|---|---|---|
| | SE + AL | RAHAR | SE + AL | RAHAR | SE + AL | RAHAR | SE + AL | RAHAR |
| Ada | 0.7489 | 0.8132 | 0.5574 | 0.6885 | 0.6966 | 0.7206 | 0.5667 | 0.9130 |
| RF | 0.8115 | 0.8746 | 0.6885 | 0.7500 | 0.7865 | 0.7647 | 0.7000 | 0.9231 |
| SVM | 0.7497 | 0.7895 | 0.3721 | 0.7077 | 0.6966 | 0.7206 | 0.6667 | 0.8519 |
| LogR | 0.5884 | 0.8649 | — | 0.6875 | — | 0.7059 | — | 0.8462 |
| Average | 0.7246 | 0.8355 | 0.5393* | 0.7154* | 0.7266* | 0.7353* | 0.6445* | 0.8960* |

*logistic regression (LogR) score is not included in averaging.

TABLE 3.2
RAHAR Health Evaluation Metrics

| | |
|---|---|
| Recall/Sensitivity | Specificity |

| | SE + AL | RAHAR | SE + AL | RAHAR |
|---|---|---|---|---|
| Ada | 0.5484 | 0.5526 | 0.7759 | 0.9333 |
| RF | 0.6774 | 0.6316 | 0.8448 | 0.9333 |
| SVM | 0.2581 | 0.6053 | 0.9310 | 0.8667 |
| LogR | — | 0.5789 | — | 0.8667 |
| Average | 0.4946* | 0.5965* | 0.8505* | 0.9111* |

*logistic regression (LogR) score is not included in averaging.

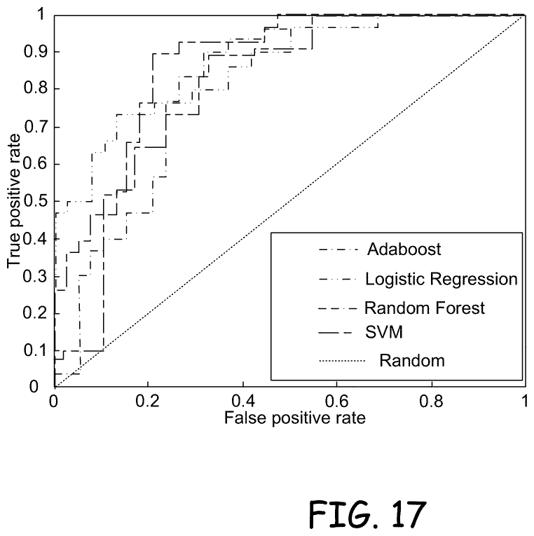

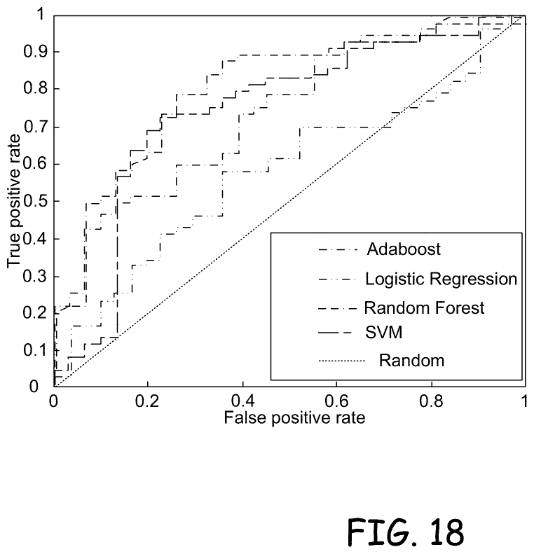

FIGS. 17 and 18 show the ROC curves for RAHAR and the sleep expert using ActiLife software (denoted as E+AL), respectively. Table 3.1 summarises the results for both RAHAR and SE+AL. One of the most important performance measures for human activity recognition is the area under the ROC curve (AU-ROC). Based on AU-ROC scores, both RAHAR and SE+AL performed best using random forest as the prediction model. Furthermore, SE+AL achieved an average AU-ROC of 0.7246 whereas RAHAR achieved 0.8355. This shows a 15% improvement of AU-ROC score, on average, by using RAHAR instead of placing the burden of manual analysis on a sleep expert. With an AU-ROC score of 0.5884 for SE+AL approach, the logistic regression model was unable to stratify the dataset, and so predicted all cases to be in a single class. This is a failure of the logistic regression model for this problem, and thus, its results are not included in this discussion. For this reason, the misleading results have also been removed from table 3.1.

Another important performance measure for human activity recognition algorithms is the F1 score. It is computed as the harmonic mean of precision and recall. According to table 3.1 and table 3.2, RAHAR performed better than SE+AL in terms of precision and recall for all models, and hence, yielded significantly higher Fl scores. Specifically, the F1 score for RAHAR, on average, was 0.7154 whereas it was 0.5393 for SE+AL (excluding logistic regression in both cases), yielding a solid margin of about 0.18 points (i.e., more than 30% improvement). On the other hand, the accuracy scores, on average, were 0.7353 for RAHAR and 0.7266 for SE+AL (again excluding logistic regression), and exhibited a relatively less significant difference still in favour of RAHAR.

### Discussion

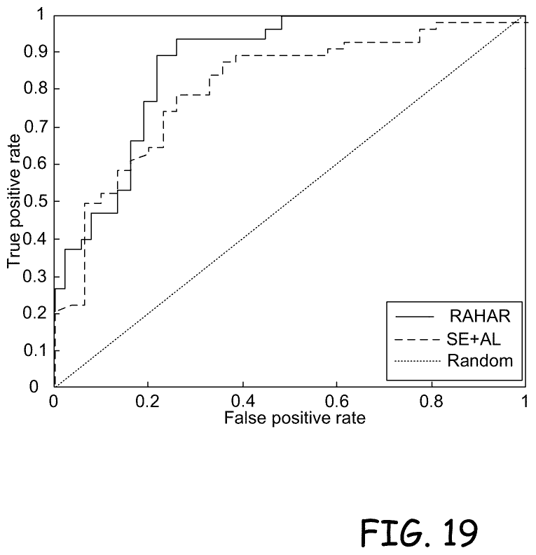

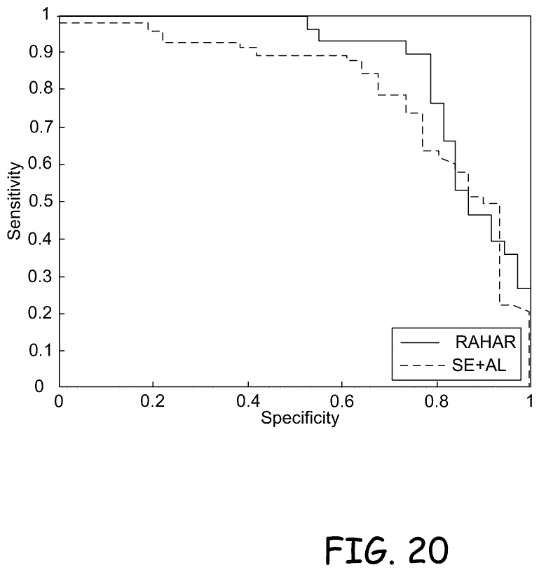

In this section we discuss the results of the best performing model and its broader impact to the area of sleep science. As seen in FIGS. 17 and 18, random forest and logistic regression were the two best performing models with the RAHAR algorithm. Based on the desired threshold value of true positive rate, TPR, (i.e., sensitivity), either model could be preferred to minimize false positive rate, FPR, (i.e., 1-specificity), which is equivalent to maximizing specificity. Random forest was also the best performing model for the SE+AL approach as mentioned earlier. If we compare the ROC as well as the sensitivity-specificity plots of the best model of each approach (i.e., random forest), we see that RAHAR outperforms SE+AL almost always as illustrated in FIGS. 19 and 20

Table 3.2 on the other hand, summarises sensitivity and specificity scores for RAHAR and SE+AL. The average sensitivity score for SE+AL and RAHAR across all models except logistic regression were 0.4946 and 0.5965, respectively. In other words, average sensitivity score for RAHAR is 20 higher than that of SE+AL. As for specificity, RAHAR with an average score of 0.9111 outperforms SE+AL with an average score of 0.8505, which corresponds to a 7% improvement.

As we seek to determine whether an individual had good quality sleep based on their physical activity levels during awake period prior to sleep, a false positive occurs when the model predicts œegood quality sleep when the person actually had a poor quality sleep. Therefore, the number of false positives needs to be kept at a minimum for a desired number of true positives. In other words, a high specificity score is sought after while keeping the sensitivity score at the desired level. As can be seen from FIG. 20 with this perspective in mind, for a large range of sensitivity scores, RAHAR achieved higher specificity scores almost all the time than SE+AL did. For example, RAHAR achieved a sensitivity score of 0.9 with a specificity score of 0.8 whereas SE+AL remained at a specificity score of 0.6 for the same sensitivity threshold.

### Conclusion

This chapter presented a robust automated human activity recognition (RAHAR) algorithm for multi-modal phenomena, and evaluated its performance in the application area of sleep science diagnostics. The results of RAHAR were tested against the results of a sleep expert using ActiLife. The goal was to predict sleep quality, specifically, sleep efficiency. RAHAR, (i) automated the activity recognition, and (ii) improved the current state-of-the-art results, on average, by 15% in terms of AU-ROC and 30% in terms of Fl scores across different models. Automating the human activity recognition puts sleep science evaluation in the hands of wearable device users. This empowers users to self-monitor their sleep-wake habits, and take action to improve the quality of their life. The improved results demonstrate the robustness of RAHAR as well as the capabilities of implementing the algorithm within clinical software such as ActiLife.

In the chapter below, the design, methodology, and retrospective evaluation for a dynamic and real-time activity recommendation system is described. This recommendation system could be used as a sleep coach within either a quantified self application, or as an automated assistant to cognitive behavioural therapy interventions.

### Approach

In order for the activity recommendation system described in this chapter to succeed, it needs to first predict the expected sleep quality at any given time t, pₜ, rather than predicting at the end of the day, p₁00. This prediction will be critical in determining what recommendation to give. If good sleep is predicted, a user may be encouraged to continue similar behaviour, but if poor sleep is predicted, a variety of possible recommendations may be provided to remedy the activity deficit.

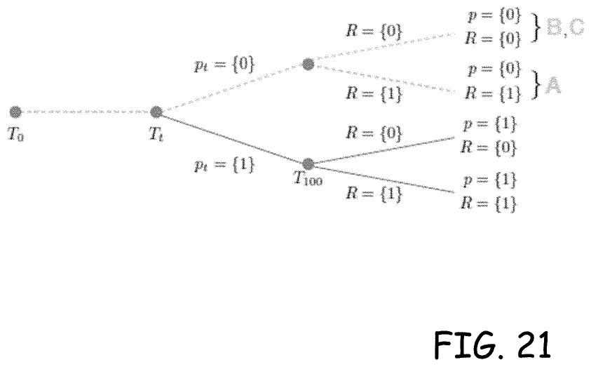

FIG. 21 illustrates the different possible predicted versus real outcomes. Activity recommendations for behaviour change would be given to individuals who are predicted to have poor sleep (pₜ={0}). This would result in a real sleep quality (R) that is either good (R={1}) or poor (R={0}).

The letter ‘A’ indicates the target situation, where an individual is predicted to have poor sleep quality (pₜ={0}), but ends up having good sleep quality (R={1}). Letters ‘B’ and ‘C’ indicate the situation where despite the early prediction of poor quality sleep, the user still resulted in having poor sleep. This could occur for two different reasons: (i) the user was non-compliant, in that they did not follow any recommendations, or (ii) the recommendation itself was faulty. Note that the recommendation would be provided at time Tₜ.

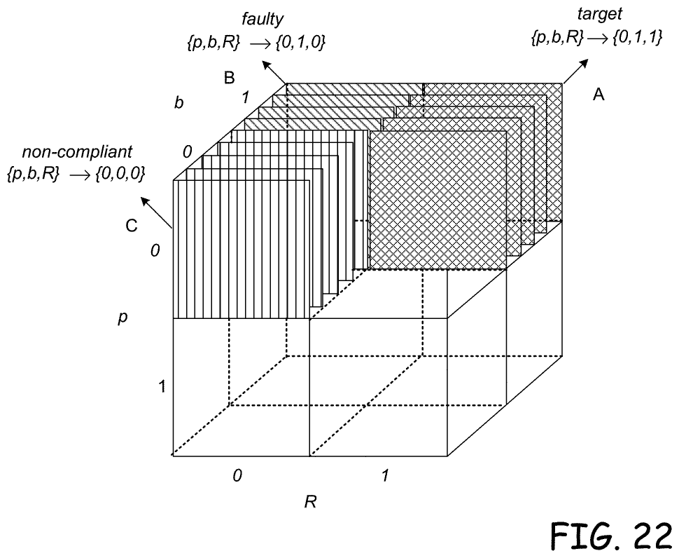

FIG. 22 shows a 3-dimensional confusion matrix with the dimensions of predicted sleep quality (p), behaviour towards recommendation (b), and the resulting sleep quality (R). Predicted sleep quality can be good or poor, (pₜ{0,1}) . The behaviour towards the recommendation is either compliant (b={1}) and the user followed the recommendation, or the user was non-compliant (b={1}) and did not follow the recommendation. Lastly, the resulting sleep quality, can also be good or poor, (R={0,1}). In a retrospective evaluation, determining the ratios in this confusion matrix can lead to insights into the effectiveness of the recommendation system and the recommendations themselves.

The details on how the recommendations are determined, are described below. The most effective and efficient way to evaluate this methodology is to do a retrospective analysis. This allows for conclusions to be drawn before a recommendation system is developed and deployed. In order to retrospectively evaluate the effectiveness of such a system, the resulting sleep quality of individuals who have compliant versus non-compliant behaviour towards recommendations should be compared. This can validate the usefulness of a recommendation, as well as the likelihood of adherence to the recommendation.

The implementation of this evaluation calls for the comparison of time series that have similar behaviour between T₀ and Tₜ₋₁, i.e. xₜ , but different behaviour from Tₜ to T₁₀₀, i.e. x₁₀₀. This is done by first clustering the activity time series, xₜ, at a time Tₜ. Thus all time series belonging to the same cluster, have exhibited similar behaviours at time Tₜ. Next is a re-clustering at time T₁₀₀ to conceive sub-clusters. Now all time series within a given sub-cluster would exhibit similar behaviour between time T₀ and T₁₀₀. Note that neighbouring sub-clusters belonging to the same outer cluster are either individuals following different recommendations, or adhering versus not adhering to the recommendations. The centroids of these sub-clusters with s minimum threshold of good-to-bad sleep quality results, make up the possible recommendations, or the behavioural recipes to achieve good sleep. Further details on this process are included below.

### Methodology

This methodology creates a dynamic activity recommendation system, and includes the necessary steps for a retrospective analysis of the potential effectiveness of those recommendations.

### Pre-Processing

The pre-processing stage can be broken down further into 2 stages: automated actigraphy and an intermediary implementation of RAHAR.

### Automated Actigraphy

The first step of pre-processing, is to run automated actigraphy on the accelerometer output. The sleep period and the awake time need to be identified to understand an individual's behaviour. Moreover, to compute the sleep efficiency, which is critical for determining what the recommendations are, as well as which recommendation to give.

### Intermediary Implementation of RAHAR

To compare the different behaviours quantitatively in terms of exertion, RAHAR can be used. RAHAR calculates the accumulated proportion of time spent in each exertion level (sedentary, light, moderate, vigorous), and can be computed at any intermediary time t.

Let xₜ represent the accumulated proportion of time spent in each of the activity levels defined in chapter 3, from time 0 to time t. In other words,

$${x}_{t} = [ \begin{matrix}{S}_{t} \\ {L}_{t} \\ {M}_{t} \\ {V}_{t}\end{matrix} ] = 〈 [ \begin{matrix}{S}_{0} \\ {L}_{0} \\ {M}_{0} \\ {V}_{0}\end{matrix} ] , [ \begin{matrix}{S}_{1} \\ {L}_{1} \\ {M}_{1} \\ {V}_{1}\end{matrix} ] , … , [ \begin{matrix}{S}_{i} \\ {L}_{i} \\ {M}_{i} \\ {V}_{i}\end{matrix} ] , … , [ \begin{matrix}{S}_{t - 1} \\ {L}_{t - 1} \\ {M}_{t - 1} \\ {V}_{t - 1}\end{matrix} ] , [ \begin{matrix}{S}_{t} \\ {L}_{t} \\ {M}_{t} \\ {V}_{t}\end{matrix} ] 〉$$

i.e. xₜ is the output of RAHAR at time t for sedentary, light, moderate, and vigorous activity, respectively. Note, The value of t will change continuously, and the RAHAR output will as well. This will lead to the recommendations dynamically updating.

### Clustering

For clustering, K-Means is used to cluster on the accumulated proportions, xₜ. However, first the number of clusters, k, must be selected. Here, the Calinski-Harabasz index (CH), also known as the variance ratio criterion, is used [T. Caliński and J. Harabasz, “A dendrite method for cluster analysis,” Communications in Statistics-theory and Methods, vol. 3, no. 1, pp. 1-27, 1974]. This method could also be substituted for other techniques as well. The test is done on one to ten clusters and iterated 1000 times. Equation 4.1, 4.2 and 4.3 define the criterion.

$$CH = \frac{BGSS / ( K - 1 )}{WGSS / ( N - K )} = \frac{N - K}{K - 1} * \frac{BGSS}{WGSS} \qquad ( 4.1 )$$

BGSS is the between-group dispersion, i.e. the weighted sum of the squared distances between cluster centroids, G^{{k}} and the overall centre, G.

$$BGSS = {\sum }_{k = 1}^{K} {n}^{k} {‖ {G}^{\{ k \}} - G ‖}^{2} \qquad ( 4.2 )$$

WGSS^{{k}} is the within cluster dispersion for cluster k. This summed over all the clusters is the WGSS, the pooled within-cluster sum of squares. M refers to the overall data matrix.

$${WGSS}^{\{ k \}} = {\sum }_{i ϵ {I}_{k}} {‖ {M}_{i}^{\{ k \}} - {G}^{\{ k \}} ‖}^{2} = \frac{1}{{n}^{k}} {\sum }_{i < j ϵ {I}_{k}} {‖ {M}_{i}^{\{ k \}} - {M}_{j}^{\{ k \}} ‖}^{2} \\ WGSS = {\sum }_{k = 0}^{K} {WGSS}^{\{ k \}} \qquad ( 4.3 )$$

The number of clusters, k, with the highest CH, is chosen. Further details on the Calinski-Harabasz index and its implementation, can be found in the original paper [T. Calński and J. Harabasz, “A dendrite method for cluster analysis,” Communications in Statistics-theory and Methods, vol. 3, no. 1, pp. 1-27, 1974]. Once the number of clusters, k has been determined k-means [T. Kanungo, D. M. Mount, N. S. Netanyahu, C. D. Piatko, R. Silverman, and A. Y. Wu, “An efficient k-means clustering algorithm: Analysis and implementation,” IEEE transactions on pattern analysis and machine intelligence, vol. 24, no. 7, pp. 881-892, 2002] is applied to all time series.

### Sub-Clustering

In the clustering discussion, the first outer level of clusters was created based on xₜ. This process is repeated within each cluster, however this time using x₁₀₀. In the case of retrospective analysis, x₁₀₀ it simply represents the remainder of a person's activity until the start of the sleep period, i.e. 100% of their activity signal. In real-time recommendations x₁00 represents xⱼ where t<j.

As a result, the outer clusters group together activity signals with similar behaviour between T₀ and Tₜ₋₁, and the sub-clusters group activity signals with similar behaviour between T₀ and Tₜ₋₁ and similar behaviour between Tₜ and T₁00 or Tⱼ.

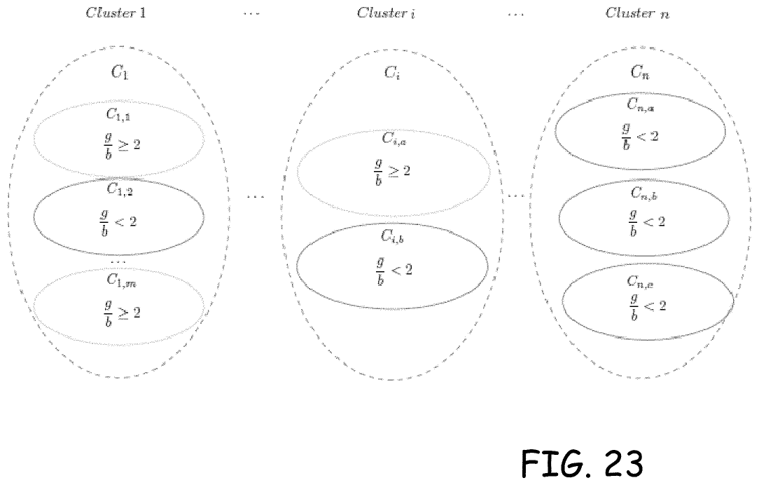

FIG. 23 provides a visualization of the sub-clustering with the lightly shaded circles denoting sub-clusters whose centroids would be behavioural recipes.

### Prediction Modelling

Before identifying the behavioural recipes, the time series need to be identified as resulting in good or poor sleep. This is determined in the pre-processing step with automated actigraphy. However, the expected sleep quality at Tₜ is also needed. If a person is expected to have poor sleep quality, the recommendation would be triggered. To determine the expected sleep quality, any of the previously defined modelling techniques mentioned above can be used.

### Identify Behavioural Recipes

To determine which of potential sub-cluster centroids can be used as a behavioural recipe, BRᵢ, the ratio of activity signals that result in good sleep relative to activity signals that result in bad sleep, is computed.

The sub-clusters that have a minimum good-to-poor ratio of or above a threshold of 2, are considered behavioural recipes. This threshold could also be modified, depending on the training population or the fidelity needs of the application area.

### Assign Recommendation

The last and final step is how to determine what recommendation to give to which user. This is computed by measuring the multi-dimensional euclidean distance between some xₜ and all BR_{i→k}. The centroid with the shortest distance to xₜ, is the chosen recommendation. As a user's behaviour is updated throughout the day, the distance simply needs to be recalculated and the selected recommendation dynamically updated. Note that the recommendations are all proportions of time spent in different activities so it would never be the case where a recommendation would imply some behaviour “undone”.

### Experimental Design

In this section, the dynamic activity recommendation system design outlined above is implemented and evaluated. The same clinical trial actigraphy data used throughout each chapter in this body of work, is used again.

### Pre-Processing

The same data described in section is used. Using the raw accelerometer output, automated actigraphy is performed to identify the relevant sleep metrics including the sleep period, sleep efficiency and sleep quality.

To run RAHAR on an intermediary time point in the dataset, we choose t as 50, where the 50 denotes 50% of the length of the time series. In otherwords:

$${x}_{50} = [ \begin{matrix}{S}_{50} \\ {L}_{50} \\ {M}_{50} \\ {V}_{50}\end{matrix} ] = 〈 [ \begin{matrix}{S}_{0} \\ {L}_{0} \\ {M}_{0} \\ {V}_{0}\end{matrix} ] , [ \begin{matrix}{S}_{1} \\ {L}_{1} \\ {M}_{1} \\ {V}_{1}\end{matrix} ] , … , [ \begin{matrix}{S}_{‖ {T}_{j} / 2 ‖} \\ {L}_{‖ {T}_{j} / 2 ‖} \\ {M}_{‖ {T}_{j} / 2 ‖} \\ {V}_{‖ {T}_{j} / 2 ‖}\end{matrix} ] 〉$$

where j is the length of the whole time series. Since each user has a different length time series, i.e. stays awake for a different amount of time, using a proportion of the awake time is more appropriate than setting a constant threshold.

### Clustering

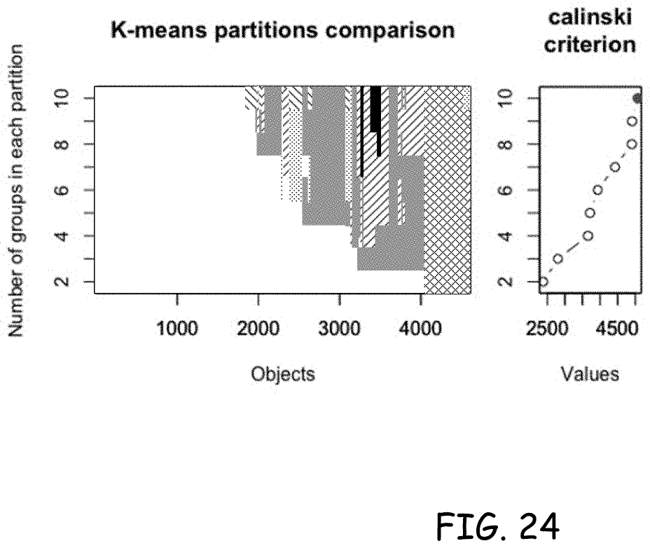

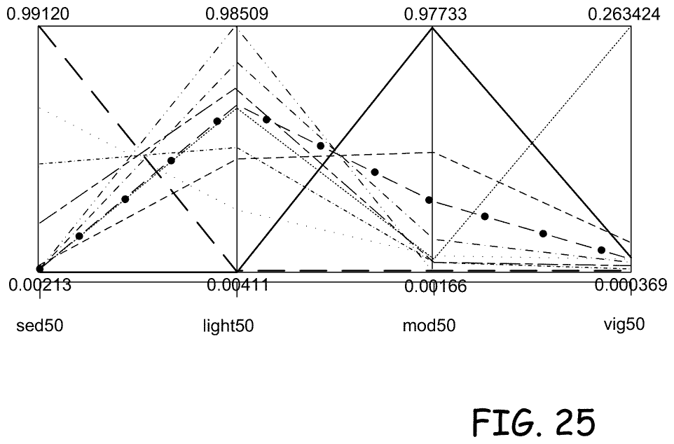

As mentioned above, the Calinski-Harabasz Index is used to determine the number of clusters to use in k-means. FIG. 24 shows a visualisation of the criterion. The highest value indicates the best number of clusters. For simplicities purpose, the maximum number of clusters is set to ten.

Based on the results seen in FIG. 24, it can be concluded that k is set to 10. Now that the number of clusters to use is determined, k-means is run. To double check the validity of the value selected for k, a cluster visualisation can be assessed. FIG. 25 is a parallel coordinates clustering visualisation, illustrating the ten clusters. The four dimensions are ‘sed50’, ‘light50’, ‘mod50’ and ‘vig50’, representing the sedentary, light, moderate and vigorous accumulated activity proportions at the point in the time series that is 50% of its total duration. The ten coloured lines zigzagging between the vertical axes represent each individual cluster. The point at which each coloured line intersects with the vertical lines indicates the value of its centroid in that dimension.

An ideal clustering illustrated in parallel coordinates will show distinctly unique lines representing each cluster. As can be seen in FIG. 25, this is the case. Although the majority of clusters seem to converge to a low value on the ‘Vig50’ axis, this is representative of the data, where the majority of individuals did not engage in vigorous activity.

### Sub-Clustering

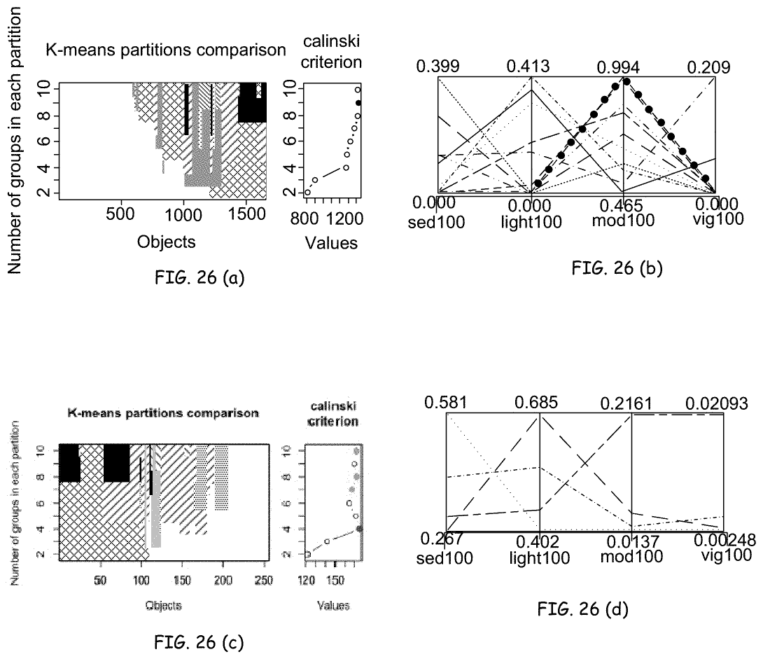

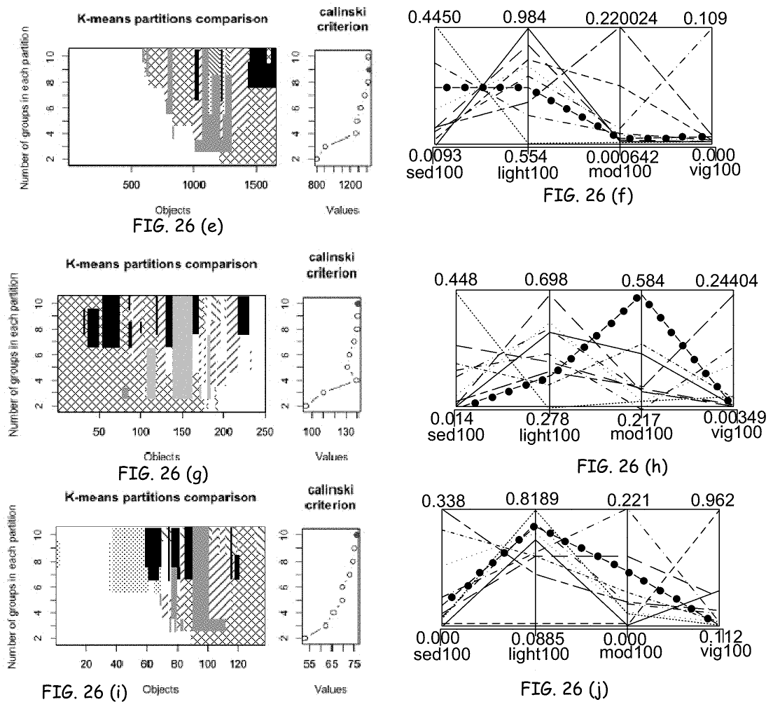

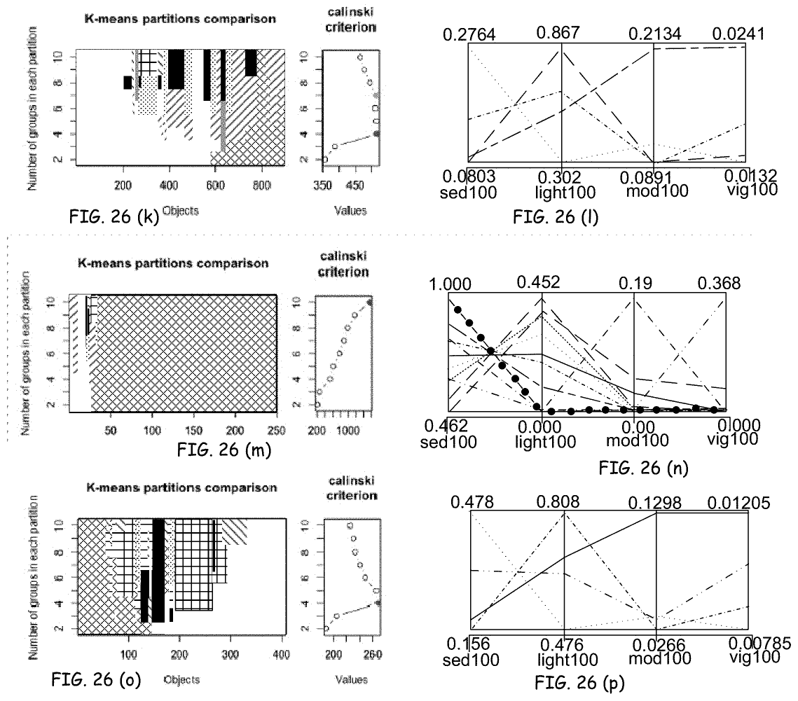

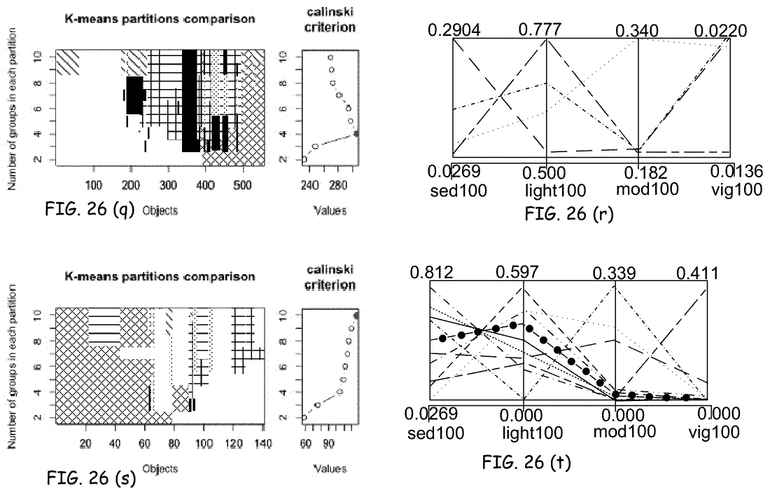

Within each of the 10 clusters, the same process is iterated: k is determined from the Calinski-Harabasz Index, and a visualisation of the cluster is completed using parallel coordinates. FIGS. 26(a)-26(t) show the graphs for each of the ten clusters.

TABLE 4.1
Optimal k for each cluster based on the Calinkski-Criterion

| Cluster | k |
|---|---|
| 1 | 9 |
| 2 | 4 |
| 3 | 9 |
| 4 | 10 |
| 5 | 10 |
| 6 | 4 |
| 7 | 10 |
| 8 | 4 |
| 9 | 4 |
| 10 | 10 |
| Total | 74 |

Table 4.1 shows a summary of the optimal k used to create the sub-clusters within each of the ten clusters. Thus the total number of sub-clusters for this dataset, is 74.

### Prediction Modelling

Any high fidelity prediction algorithm can be used to predict the sleep quality at T₅₀. For simplicities sake, random forest is used here. Table 4.2 shows a confusion matrix of the predicted versus real sleep quality. Note that all individuals predicted to have poor sleep would receive a recommendation, i.e. 869 of the 3226 individuals.

TABLE 4.2
A Confusion Matrix of Predicted v. Real Sleep Quality

| | Predicted at T₅₀ | | |
|---|---|---|---|
| Observed | Good {1} | Poor {0} | Total |
| Good {1} | 1692 | 107 | 1799 |
| Poor {0} | 659 | 762 | 1421 |
| Total | 2351 | 869 | 3226 |

### Identify Behavioural Recipes

At this point, the sub-clusters have been defined, as well as the predicted and observed sleep quality for each time series. Thus the ratio of the observed, or real (R), good to bad sleep ratio can be computed for each sub-cluster. Tables 4.3, 4.4, 4.5, 4.6, 4.7, 4.8, 4.9, 4.10, 4.11 and 4.12 show the proportions and ratios for each of the corresponding sub-clusters. The centroid of any sub-cluster with a ratio greater than a threshold of 2, is considered a behavioural recipe.

TABLE 4.3
Good/Bad sleep ratios for each sub-cluster in Cluster 1

| | Cluster | | | | | | | | | |
|---|---|---|---|---|---|---|---|---|---|---|
| | 1 | 1 | 1 | 1 | 1 | 1 | 1 | 1 | 1 | 1 |
| | Sub-Cluster | | | | | | | | | |
| | 1 | 2 | 3 | 4 | 5 | 6 | 7 | 8 | 9 | 10 |
| Prop. of Poor Quality | 0.25 | 0.00 | 0.00 | 1.00 | 0.22 | 0.25 | 0.67 | 0.33 | 0.50 | 0.33 |
| Prop. of Good Quality | 0.75 | 1.00 | 1.00 | 0.00 | 0.78 | 0.75 | 0.33 | 0.67 | 0.5 | 0.67 |
| Ratio | 3 | ∞ | ∞ | 0.00 | 3.50 | 3.00 | 0.5 | 2.00 | 1.00 | 2.00 |

TABLE 4.4
Good/Bad sleep ratios for each sub-cluster in Cluster 2

Cluster

| | 2 | 2 | 2 | 2 |
|---|---|---|---|---|
| Sub-Cluster | 1 | 2 | 3 | 4 |
| Prop. of Poor Quality | 0.55 | 0.56 | 0.34 | 0.76 |
| Prop. of Good Quality | 0.45 | 0.44 | 0.66 | 0.24 |
| Ratio | 0.83 | 0.79 | 1.93 | 0.32 |

TABLE 4.5
Good/Bad sleep ratios for each sub-cluster in Cluster 3

| | Cluster | | | | | | | | |
|---|---|---|---|---|---|---|---|---|---|
| | 3 | 3 | 3 | 3 | 3 | 3 | 3 | 3 | 3 |
| | Sub-Cluster | | | | | | | | |
| | 1 | 2 | 3 | 4 | 5 | 6 | 7 | 8 | 9 |
| Prop. of Poor Quality | 0.51 | 0.26 | 0.39 | 0.62 | 0.45 | 0.61 | 0.49 | 0.16 | 0.56 |
| Prop. of Good Quality | 0.49 | 0.74 | 0.61 | 0.38 | 0.55 | 0.39 | 0.651 | 0.84 | 0.44 |
| Ratio | 0.94 | 2.85 | 1.60 | 0.62 | 1.21 | 0.63 | 1.06 | 5.22 | 0.80 |

TABLE 4.6
Good/Bad sleep ratios for each sub-cluster in Cluster 4

| | Cluster | | | | | | | | | |
|---|---|---|---|---|---|---|---|---|---|---|
| | 4 | 4 | 4 | 4 | 4 | 4 | 4 | 4 | 4 | 4 |
| | Sub-Cluster | | | | | | | | | |
| | 1 | 2 | 3 | 4 | 5 | 6 | 7 | 8 | 9 | 10 |
| Prop. of Poor Quality | 0.53 | 0.71 | 0.58 | 0.29 | 0.07 | 0.29 | 1.00 | 0.38 | 0.31 | 0.90 |
| Prop. of Good Quality | 0.47 | 0.29 | 0.42 | 0.71 | 0.93 | 0.71 | 0.00 | 0.62 | 0.69 | 0.10 |
| Ratio | 0.89 | 0.41 | 0.71 | 2.5 | 14 | 2.43 | 0.00 | 1.60 | 2.21 | 0.11 |

TABLE 4.7
Good/Bad sleep ratios for each sub-cluster in Cluster 5

| | Cluster | | | | | | | | | |
|---|---|---|---|---|---|---|---|---|---|---|
| | 5 | 5 | 5 | 5 | 5 | 5 | 5 | 5 | 5 | 5 |
| | Sub-Cluster | | | | | | | | | |
| | 1 | 2 | 3 | 4 | 5 | 6 | 7 | 8 | 9 | 10 |
| Prop. of Poor Quality | 0.90 | 0.00 | 0.22 | 0.83 | 0.16 | 0.50 | 0.80 | 0.57 | 0.40 | 0.45 |
| Prop. of Good Quality | 0.10 | 1.00 | 0.78 | 0.17 | 0.84 | 0.50 | 0.20 | 0.43 | 0.60 | 0.55 |
| Ratio | 0.11 | ∞ | 3.50 | 0.20 | 5.33 | 1.00 | 0.25 | 0.75 | 1.50 | 1.2 |

TABLE 4.8
Good/Bad sleep ratios for each sub-cluster in Cluster 6

Cluster

| | 6 | 6 | 6 | 6 |
|---|---|---|---|---|
| Sub-Cluster | 1 | 2 | 3 | 4 |
| Prop. of Poor Quality | 0.59 | 0.30 | 0.25 | 0.75 |
| Prop. of Good Quality | 0.41 | 0.70 | 0.75 | 0.25 |
| Ratio | 0.70 | 2.33 | 2.98 | 0.33 |

TABLE 4.9
Good/Bad sleep ratios for each sub-cluster in Cluster 7

| | Cluster | | | | | | | | | |
|---|---|---|---|---|---|---|---|---|---|---|
| | 7 | 7 | 7 | 7 | 7 | 7 | 7 | 7 | 7 | 7 |
| | Sub-Cluster | | | | | | | | | |
| | 1 | 2 | 3 | 4 | 5 | 6 | 7 | 8 | 9 | 10 |
| Prop. of Poor Quality | 0.50 | 0.00 | 0.00 | 1.00 | 0.73 | 0.33 | 0.50 | 0.33 | 0.67 | 0.50 |
| Prop. of Good Quality | 0.50 | 1.00 | 1.00 | 0.00 | 0.27 | 0.67 | 0.50 | 0.67 | 0.33 | 0.50 |
| Ratio | 1.00 | ∞ | ∞ | 0.00 | 3.67 | 2.00 | 1.00 | 2.00 | 0.50 | 1.00 |

TABLE 4.10
Good/Bad sleep ratios for each sub-cluster in Cluster 8

Cluster

| | 8 | 8 | 8 | 8 |
|---|---|---|---|---|
| Sub-Cluster | 1 | 2 | 3 | 4 |
| Prop. of Poor Quality | 0.41 | 0.62 | 0.51 | 0.82 |
| Prop. of Good Quality | 0.59 | 0.38 | 0.49 | 0.18 |
| Ratio | 1.42 | 0.62 | 0.94 | 0.23 |

TABLE 4.11
Good/Bad sleep ratios for each sub-cluster in Cluster 9

Cluster

| | 9 | 9 | 9 | 9 |
|---|---|---|---|---|
| Sub-Cluster | 1 | 2 | 3 | 4 |
| Prop. of Poor Quality | 0.59 | 0.24 | 0.57 | 0.37 |
| Prop. of Good Quality | 0.41 | 0.76 | 0.43 | 0.62 |
| Ratio | 0.68 | 3.13 | 0.77 | 1.65 |

TABLE 4.12
Good/Bad sleep ratios for each sub-cluster in Cluster 10

| | Cluster | | | | | | | | | |
|---|---|---|---|---|---|---|---|---|---|---|
| | 10 | 10 | 10 | 10 | 10 | 10 | 10 | 10 | 10 | 10 |
| | Sub-Cluster | | | | | | | | | |
| | 1 | 2 | 3 | 4 | 5 | 6 | 7 | 8 | 9 | 10 |
| Prop. of Poor Quality | 0.88 | 0.33 | 0.46 | 0.00 | 0.53 | 0.60 | 1.00 | 0.33 | 0.75 | 0.83 |
| Prop. of Good Quality | 0.13 | 0.67 | 0.54 | 1.00 | 0.47 | 0.40 | 0.00 | 0.67 | 0.25 | 0.17 |
| Ratio | 0.14 | 2.00 | 1.17 | ∞ | 0.88 | 0.67 | 0.00 | 2.00 | 0.33 | 0.20 |

### Determine Recommendation

For real-time recommendations, the multidimensional euclidean distance from an individual's xₜ to all of the centroids included in table 4.13 is computed. The shortest distance indicates the chosen recommendation for suggestion.

From the above table it is clear that not all clusters contain a behavioural recipe. This is an issue for a retrospective evaluation where it is necessary to do a comparison on the resulting sleep quality of people who have the same predicted sleep quality (p₅₀), but either do or do not follow the recommendation. Thus any time series in a cluster that does not contain a behavioural recipe is reassigned. The reassignment is determined by measuring the distance from xₜ to each of the centroids. This is the same process as the recommendation decision, and essentially is determining what recommendation would be given to that individual.

TABLE 4.13
The Behavioural Recipes

| Cluster | Subcluster | Sedentary | Light | Moderate | Vigorous |
|---|---|---|---|---|---|
| 1 | 1 | 0.010 | 0.413 | 0.577 | 0.000 |
| 1 | 5 | 0.004 | 0.002 | 0.994 | 0.00 |
| 1 | 6 | 0.266 | 0.000 | 0.734 | 0.000 |
| 3 | 2 | 0.024 | 0.864 | 0.108 | 0.003 |
| 3 | 8 | 0.009 | 0.984 | 0.005 | 0.002 |
| 4 | 4 | 0.479 | 0.415 | 0.293 | 0.244 |
| 4 | 6 | 0.227 | 0.698 | 0.273 | 0.006 |
| 4 | 9 | 0.027 | 0.561 | 0.397 | 0.015 |
| 5 | 5 | 0.066 | 0.715 | 0.107 | 0.112 |
| 6 | 2 | 0.045 | 0.718 | 0.213 | 0.024 |
| 6 | 3 | 0.030 | 0.867 | 0.089 | 0.014 |
| 9 | 2 | 0.027 | 0.777 | 0.812 | 0.014 |

### Evaluate Recommendation

In order to evaluate whether a recommendation was taken, a comparison needs to be done on the resulting sleep quality of people who have the same predicted sleep quality (p₅₀), but either (i) follow the recommendation (b={1}), or (ii) do not (b={0}).

Two activity signals belonging to the same cluster, but different sub-clusters, indicates that the signals followed similar behaviours between T₀ and T₅₀, and different behaviours between T₅₀ and T₁₀₀. Assume both of these activity signals were also predicted to have poor sleep, i.e. they received a recommendation. The signals that belong to a sub-cluster with a good-to-bad ratio greater than 2, are in the same cluster as a behaviour recipe. This suggests that they exhibit similar behaviour to the recommendation, so it naturally concludes that they followed, or adhered, to the recommendation. Conversely, it can be concluded that any signals belonging to sub-clusters with a good-to-bad ratio below 2, did not adhere to the recommendation, and were non-compliant.

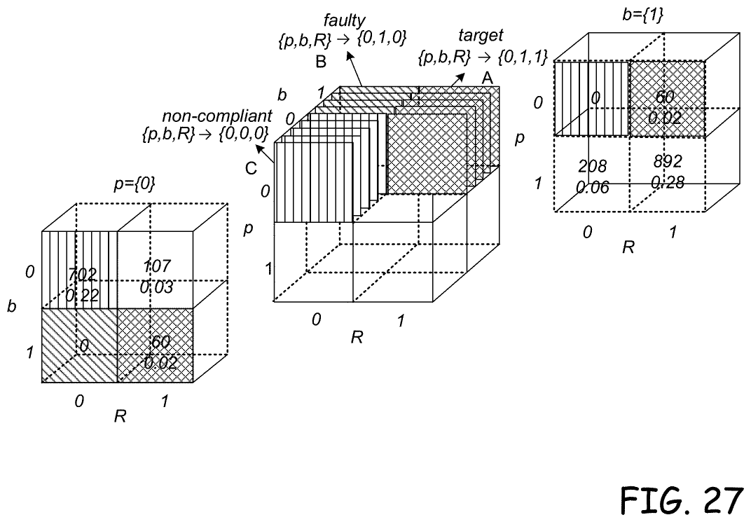

FIG. 27 contains summary results from the experiment. Note that in this dataset the majority of activity signals are predicted to have good sleep at T₅₀, and do indeed have good sleep. This is one of the causes of such low proportions visible in the confusion matrix.

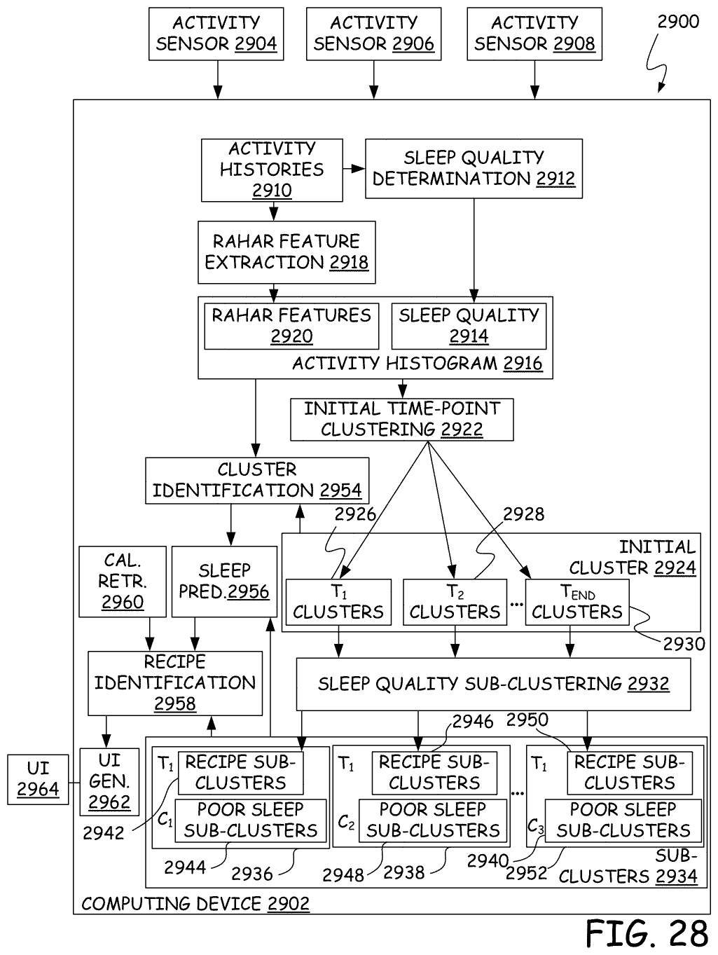

FIG. 28 provides a block diagram of a system 2900 used to implement the various embodiments discussed above. System 2900 includes a computing device 2902 and a plurality of activity sensors, such as activity sensors 2904, 2906 and 2908. Each activity sensor is worn by or carried by a different person and provides activity sensor values indicative of movement of the person. The activity sensor values are provided to computing device 2902. The activity sensor values are stored in computing device 2902 as activity histories 2910. In accordance with one embodiment, each activity history 2910 is for a single day for a single person. Each activity history 2910 covers an active period of the day and a sleeping period of the day.

Activity histories 2910 are provided to a sleep quality determination module 2912, which uses the automated actigraphy discussed above to identify a sleep period, sleep onset, awake time, time to bed, latency, WASO and time to active. These values are then used to identify a sleep quality 2914 represented by the activity in the particular activity history 2910. Activity history 2910 is also provided to a RAHAR feature extraction module 2918, which segments the activity history 2910 by using changepoint detection as discussed above and then identifies an exertion level for each segment of activity history 2910. This produces RAHAR features 2920. Since the changepoint detection is different for each user on each day, the number of different exertion levels in RAHAR features 2920 and the time points where those exertion levels change will be different for each user and for each day of activity for a single user.

RAHAR features 2920 and sleep quality 2914 together form activity histogram 2916 for a particular person on a particular day. In particular, activity histogram 2916 consists of a sequence of exertion levels and an associated sleep quality for a particular day.

Activity histograms 2916 are provided to an initial time point clustering module 2922, which clusters the activity histograms 2916 at each of a plurality of time points to form time point cluster groupings, such as time point cluster groupings 2926, 2928 and 2930, which together form initial clusters 2924. Each of cluster groupings 2926, 2928 and 2930 include all of the activity histograms 2916. The difference between the cluster groupings is that the clustering is only performed based on the RAHAR features 2920 up to the time point associated with those clusters. For example, the T₁ clusters 2926 only uses RAHAR features in activity histograms 2916 up to time point T₁ while the T₂ clusters are clustered based on RAHAR features 2920 up to time T₂, which is later than time T₁. T₁ clusters 2926, T₂ clusters 2928, and Tₙ clusters 2930 all contain a number of different clusters of activity histograms 2916. In accordance with one embodiment, each of the cluster groupings 2926, 2928 and 2930 contain the same number of clusters.

Each of the cluster groupings 2926, 2928 and 2930 is provided to a sleep quality sub-clustering module 2932, which forms separate sub-clusters for each cluster in each cluster grouping. For example, for T₁ cluster C₁ 2936, recipe sub-clusters 2942 and poor sleep sub-clusters 2944 are formed by sleep quality sub-clustering module 2932. For T₁ cluster C₂ 2938, recipe sub-clusters 2946 and poor sleep sub-clusters 2948 are formed and for T₁ cluster C₃ 2940, recipe sub-clusters 2950 and poor sleep sub-cluster 2952 are formed by sleep quality sub-clustering module 2932. Similar sub-clusters are formed for each of the clusters in T₂ clusters 2928 and Tₙ clusters 2930. The sub-clusters identified by sleep quality sub-clustering module 2932 are formed based on the the entire pre-sleep period of each activity histogram in the respective cluster. Thus, where the cluster was formed based on a partial activity histogram up to some time, such as T₁ or T₂, the sub-clusters are formed based on the full histogram from the start of activity until the end of activity where the end of activity is identified by the start of sleep. After the sub-clusters are formed, they are designated as either recipe sub-clusters or as poor sleep sub-clusters. A recipe sub-cluster is a sub-cluster in which a majority of the activity histograms assigned to the sub-cluster resulted in good sleep while in poor sleep sub-clusters, the majority of assigned histograms resulted in poor sleep.

Once the sub-clusters have been formed, they can be used together with the initial clusters 2924 to predict whether an in-progress activity histogram is likely to result in poor sleep and if so, to recommend activities to be performed to increase the probability of achieving good sleep at the end of the day.

The in-progress activity histogram is formed from activity values generated by an activity sensor, such as one of activity sensors 2904, 2906 and 2908. The activity values are provided to RAHAR feature extractions 2918 to identify RAHAR features up to the current time point representing the in-progress activity histogram. The in-progress activity histogram is provided to a cluster identification module 2954, which identifies a closest initial cluster 2924 of the clusters formed for the end time point of the in-progress activity histogram. For example, if the in-progress activity histogram ends at time T₂, the closest cluster in T₂ clusters 2928 would be identified, whereas if the in-progress activity histogram ends at time T₁, the closet cluster in T₁ clusters 2926 would be identified.

A sleep prediction module 2956 then predicts whether the in-progress activity histogram is likely to result in poor sleep or good sleep by identifying the closest sub-cluster within the closest cluster identified by cluster identification module 2954. If the closet sub-cluster is part of a poor sleep sub-cluster, then the in-progress activity histogram is likely to result in poor sleep whereas if the closest sub-cluster is part of a recipe sub-cluster, the activity histogram is likely to result in good sleep. In identifying a closest sub-cluster, only the features in each sub-cluster centroid up to the ending time of the in-progress activity histogram are used to determine the distance between the in-progress activity histogram and the respective sub-cluster centroid.

If the closest sub-cluster identified by sleep prediction module 2956 is a recipe sub-cluster, either no action can be taken or a user interface can be generated to inform the user that they are likely to enjoy good sleep at the end of the day. If the in-progress activity histogram is closest to a poor sleep sub-cluster, a recipe identification module 2958 identifies activities that the user can perform to improve the probability of enjoying a good night sleep. To identify an activity, recipe identification module 2958 searches for the closest recipe sub-cluster in the cluster identified by cluster identification 2954 for the in-progress activity histogram. The identification of the closest recipe sub-cluster is performed by determining the distance between the feature sequence of the in-progress activity histogram to the centroids of each recipe sub-cluster using only the sequences of RAHAR features up to the end time of the in-progress activity histogram. Thus, the entire centroid of the recipe sub-clusters are not used in identifying the closest recipe sub-cluster.

Once the closet recipe sub-cluster has been identified, the centroid of the closest recipe sub-cluster is used to make an activity recommendation. In accordance with one embodiment, the RAHAR features of the centroid at any time point are proportions of exertion levels that have occurred up to that time point. To identify what activity to recommend, the proportion of the various exertion levels at the end of the selected recipe sub-cluster (just prior to sleep starting) is compared to the latest proportions of exertion levels in the in-progress activity histogram. The differences between the exertion levels are then used to identify particular exertion levels that should be increased or decreased by the user. Activities that will achieve the desired porportion of activity levels are then identified by recipe identification unit 2958, such as running, walking and resting. These identified activities are then provided to a user interface generation module 2962 to generate a user interface 2964 that can be perceived by the user and that will include the identified activity. In accordance with one embodiment, recipe identification module 2958 uses a calendar retrieval module 2960 to retrieve calendar entries for the user for the current day. These calendar entries are then used to identify available time periods when the user can perform activities. The activity recommended to the user is then selected based on the available time that the user has in the remainder of the day. For example, if possible activities included walking for a half-hour or running for 15 minutes, and the person only had 15 minutes available, the suggested activity would be to run for 15 minutes instead of walking for a half-hour.

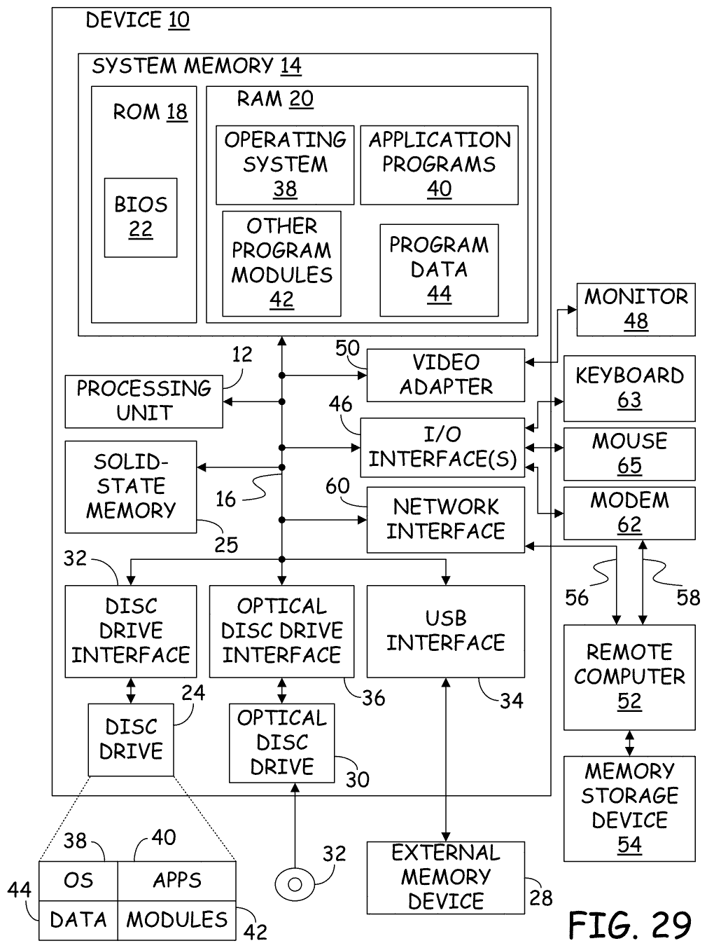

FIG. 29 provides an example of a computing device 10 that can be used as a server or client device in in the embodiments above. Computing device 10 includes a processing unit 12, a system memory 14 and a system bus 16 that couples the system memory 14 to the processing unit 12. System memory 14 includes read only memory (ROM) 18 and random access memory (RAM) 20. A basic input/output system 22 (BIOS), containing the basic routines that help to transfer information between elements within the computing device 10, is stored in ROM 18. Computer-executable instructions that are to be executed by processing unit 12 may be stored in random access memory 20 before being executed.

Embodiments of the present invention can be applied in the context of computer systems other than computing device 10. Other appropriate computer systems include handheld devices, multi-processor systems, various consumer electronic devices, mainframe computers, and the like. Those skilled in the art will also appreciate that embodiments can also be applied within computer systems wherein tasks are performed by remote processing devices that are linked through a communications network (e.g., communication utilizing Internet or web-based software systems). For example, program modules may be located in either local or remote memory storage devices or simultaneously in both local and remote memory storage devices. Similarly, any storage of data associated with embodiments of the present invention may be accomplished utilizing either local or remote storage devices, or simultaneously utilizing both local and remote storage devices.

Computing device 10 further includes an optional hard disc drive 24, an optional external memory device 28, and an optional optical disc drive 30. External memory device 28 can include an external disc drive or solid state memory that may be attached to computing device 10 through an interface such as Universal Serial Bus interface 34, which is connected to system bus 16. Optical disc drive 30 can illustratively be utilized for reading data from (or writing data to) optical media, such as a CD-ROM disc 32. Hard disc drive 24 and optical disc drive 30 are connected to the system bus 16 by a hard disc drive interface 32 and an optical disc drive interface 36, respectively. The drives and external memory devices and their associated computer-readable media provide nonvolatile storage media for the computing device 10 on which computer-executable instructions and computer-readable data structures may be stored. Other types of media that are readable by a computer may also be used in the exemplary operation environment.

A number of program modules may be stored in the drives and RAM 20, including an operating system 38, one or more application programs 40, other program modules 42 and program data 44. In particular, application programs 40 can include programs for implementing any one of modules discussed above. Program data 44 may include any data used by the systems and methods discussed above.

Processing unit 12, also referred to as a processor, executes programs in system memory 14 and solid state memory 25 to perform the methods described above.

Input devices including a keyboard 63 and a mouse 65 are optionally connected to system bus 16 through an Input/Output interface 46 that is coupled to system bus 16. Monitor or display 48 is connected to the system bus 16 through a video adapter 50 and provides graphical images to users. Other peripheral output devices (e.g., speakers or printers) could also be included but have not been illustrated. In accordance with some embodiments, monitor 48 comprises a touch screen that both displays input and provides locations on the screen where the user is contacting the screen.

The computing device 10 may operate in a network environment utilizing connections to one or more remote computers, such as a remote computer 52. The remote computer 52 may be a server, a router, a peer device, or other common network node. Remote computer 52 may include many or all of the features and elements described in relation to computing device 10, although only a memory storage device 54 has been illustrated in FIG. 29. The network connections depicted in FIG. 29 include a local area network (LAN) 56 and a wide area network (WAN) 58. Such network environments are commonplace in the art.

The computing device 10 is connected to the LAN 56 through a network interface 60. The computing device 10 is also connected to WAN 58 and includes a modem 62 for establishing communications over the WAN 58. The modem 62, which may be internal or external, is connected to the system bus 16 via the I/O interface 46.

In a networked environment, program modules depicted relative to the computing device 10, or portions thereof, may be stored in the remote memory storage device 54. For example, application programs may be stored utilizing memory storage device 54. In addition, data associated with an application program may illustratively be stored within memory storage device 54. It will be appreciated that the network connections shown in FIG. 29 are exemplary and other means for establishing a communications link between the computers, such as a wireless interface communications link, may be used.

Although elements have been shown or described as separate embodiments above, portions of each embodiment may be combined with all or part of other embodiments described above.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms for implementing the claims.

What is claimed is:

1. A method comprising:
associating a person's activities up to a current time point with one of a plurality of clusters of activity proportions for the current time point;
associating the person's activities up to the current time point with a sub-cluster in the cluster of activity proportions for the current time point;
determining that the sub-cluster is associated with poor sleep; and
recommending at least one activity to increase the likelihood of good sleep.

2. The method of claim 1 wherein recommending at least one activity to increase the likelihood of good sleep comprises:
identifying a sub-cluster in the cluster that is associated with good sleep;
retrieving exertion levels for the sub-cluster from the current time to an expected time to go to sleep; and
recommending the at least one activity based on the retrieved exertion levels.

3. The method of claim 2 wherein retrieving exertion levels from the sub-cluster comprises retrieving exertion levels from the centroid of the sub-cluster.

4. The method of claim 1 wherein associating the person's activities up to the current time point with a sub-cluster in the cluster of activity proportions for the current time point comprises selecting a closest sub-cluster based on the person's activity up to the current time point.

5. The method of claim 1 wherein recommending at least one activity comprises retrieving the person's calendar and determining what activities can be performed before it is time to sleep given the entries in the person's calendar.

6. The method of claim 1 wherein determining that a sub-cluster is associated with poor sleep comprises:
for a plurality of people, collecting activity measures from a respective activity sensor over an entire day;
using the activity measures to estimate when the person fell asleep and the quality of the person's sleep;
forming the clusters and sub-clusters based on the activity measures; and
using the quality of each person's sleep in each sub-cluster to determine whether the sub-cluster is associated with poor sleep.

7. A method comprising:
clustering partial activity histograms, wherein each partial activity histogram extends from a start of activities to a selected time after the start of activities;
for each cluster, forming sub-clusters of full activity histograms, wherein each full activity histogram extends from a start of activities to an end of activities;
for each sub-cluster, identifying a most-likely outcome of the full activity histograms in the sub-cluster; and
receiving an in-progress activity histogram extending from the start of activities to the selected time after the start of activities and identifying a most-likely outcome for the in-progress activity histogram by assigning the in-progress activity histogram to one of the sub-clusters.

8. The method of claim 7 further comprising:
from the sub-clusters, identifying recipe sub-clusters having a most-likely outcome that is considered a desired outcome; and
using the in-progress activity histogram to select one of the recipe sub-clusters; and
using the selected recipe sub-cluster to suggest at least one activity to be performed to reach a desired outcome.

9. The method of claim 8 wherein each recipe sub-cluster is defined by a respective centroid representing a full activity histogram.

10. The method of claim 9 wherein using the recipe sub-cluster to suggest at least one activity to be performed to reach a desired outcome comprises identifying the at least one activity to be performed by comparing the full activity histogram of the recipe sub-cluster's centroid to the in-progress activity histogram.

11. The method of claim 10 wherein using the recipe sub-cluster to suggest at least one activity to be performed further comprises retrieving a calendar of scheduled activities and suggesting an additional activity that can be completed given the calendar of scheduled activities.

12. The method of claim 7 wherein the most-likely outcome comprises good sleep or bad sleep.

13. The method of claim 12 wherein each partial activity histogram comprises proportions of exertion levels from the start of activities to the selected time and the in-progress activity histogram comprises proportions of exertion levels from the start of activities to the selected time.

14. The method of claim 13 wherein each full activity histogram comprises proportions of exertion levels from the start of activities to the end of activities.

15. The method of claim 7 wherein each full activity histogram comprises a separately determined division of activities from all other full activity histograms.

16. A system comprising:
an activity sensor providing periodic activity values;
a sleep prediction module, predicting at multiple times per day a quality of sleep that is expected at the end of the day based on the periodic activity values.

17. The system of claim 16 wherein the predicted quality of sleep changes over the course of the day as new activity values are received.

18. The system of claim 17 wherein the quality of sleep is predicted when there is sufficient time to perform at least one activity that will make it more likely that the quality of sleep will be good.

19. The system of claim 18 further comprising a recipe identification module that identifies the at least one activity that will make it more likely that the quality of sleep will be good by identifying a cluster of training activity histograms that is similar to the periodic activity values.

20. The system of claim 19 wherein the at least one activity is based on the centroid of the identified cluster.
